(19) United States
(12) Patent Application Publication
(10) Pub. No.: US 2004/0267283 A1
(43) Pub. Date: Dec. 30, 2004

(54) Method, device and kit for body decoration

(76) Inventors: Daniela Mavor, Tel Aviv (IL); Zvi Nitzan, Zofit (IL); Nurit Harel, Tel Aviv (IL); Michal Shahar, Rishon LeZion (IL); Giora Arbel, Kfar Saba (IL); Yosef Gross, Moshav Mazor (IL); Dalia Jayes, Modiin (IL); Dov Tamarkin, Maccabim (IL)

Correspondence Address: KENYON & KENYON, 1500 K STREET, N.W., SUITE 700, WASHINGTON, DC, 20005, US

(21) Appl. No.: 10/821,966

(22) Filed: Apr. 12, 2004

Related U.S. Application Data
(60) Provisional application No. 60/482,018, filed on Jun. 25, 2003.

(51) Int. Cl. A61B 17/00

(52) U.S. Cl. 606/116; 81/9.22; 604/20

(57) ABSTRACT

A method, device and kit for body decoration. Embodiments of the kit include an ink suitable for body decoration, means for applying the ink to a desired body area, and an electrically powered patch detachably coupled to the application means to promote penetration of the ink into the body area. Embodiments of the device include a fully integrated patch device. The patch device integrates the elements of a kit (i.e., a color formulation, a means for applying, and a patch) into a stand-alone patch. Embodiments of the method include contacting the desired body area with a means for applying, applying ink through the means for applying, and promoting penetration of the ink into the body area with the patch by applying electric current to the ink application. Exemplary applications include temporary and permanent tattooing, applying semi-permanent cosmetics, animal tattooing, and administering therapeutic treatments.

### CROSS REFERENCE TO RELATED APPLICATION

This application claims priority to U.S. Provisional Application No. 60/482,018, filed Jun. 25, 2003, the contents of which are incorporated herein by reference.

### FIELD OF THE INVENTION

The present invention relates in general to body decorating and in particular to body decorating using electric current enhancement.

### BACKGROUND

Body decoration, such as tattooing, is an ancient form of art, which has been known and used for thousands of years. Today, body decoration is a growing business, driven by both the adolescent population and their lifestyle trends, as well as by older consumers. Various methods of dyeing or tattooing the skin have been employed for centuries. For example, one technique is simply painting the skin. However, this requires a certain degree of artistic ability and an understanding of various paints or dyes and their effect on skin. Tattooing typically involves dyeing the skin using needles and permanent dyes. However, this is a painful process, and permanent tattoos are often seen as undesirable due to their permanent nature, their cost, the pain involved, the possible health risks associated with contaminated needles and the need for a skilled tattoo artist.

As a remedy to the inalterable permanence of the tattoo art, several technologies have recently become available that allow tattoos to be removed, but these processes are painful, expensive, and relatively slow, often requiring multiple treatments to achieve only moderate success. In addition, tattoos are currently provided by specialized tattoo parlors, adding to their cost and inaccessibility. As a result of the associated deficiencies of permanent tattoos and the available removal techniques as outlined hereinabove, many people who are interested in personal expression and body art are dissuaded from getting a tattoo.

Temporary tattoos are also available, such as tattoos from henna and stick on tattoos. However, temporary tattoos suffer from the disadvantages of being very short-lived and relatively limited in the variety of possible images as they are manufactured in a finite set of popular likenesses. Further, temporary tattoos can cause allergic irritation due to the colors used in such tattoos.

Iontophoresis has been known for many years as a means to deliver drugs or other substances into the skin. It is based on known mechanisms, including (a) iontophoresis, in which a charged ion is repelled from an electrode of the same charge, and (b) electroosmosis, based on the convective movement of solvent that occurs through a charged “pore” in response to the preferential passage of counter-ions when the electric field is applied. For example, Mishima, in U.S. Pat. No. 5,262,153, describes a method for whitening the skin by administering a whitening agent via iontophoresis. Jacobsen, in U.S. Pat. No. 4,141,359, discloses the introduction of drugs via iontophoresis in conjunction with the marking of the drug entry skin area with ink. Oester in “Iontophoresis With Dye Substances, Inorganic Compound and Organic Drugs: Experimental Studies,” Arch. Phys. Med. Rehab., Vol.34, October 1953, pp. 627-633, describes experiments performed on laboratory rats to determine the degree to which substances penetrate the body via iontophoresis. Since iontophoresis is a painless, non-invasive technique for delivering substances into the body, it provides an attractive alternative to the existing tattooing approaches.

Accordingly, iontophoresis has been suggested as a means for tattooing the skin. For example, Eppstein, in U.S. Pat. Nos. 5,445,611, suggests tattooing via iontophoresis. Henley, in U.S. Pat. No. 6,477,410, also suggests iontophoretic delivery of a substance to create or remove a tattoo. However, neither discloses the information necessary to enable one of ordinary skill in the art to actually apply or remove a tattoo using iontophoresis, much less the information necessary to make a device or kit that can be employed by a layman to apply or remove a tattoo using iontophoresis.

Other methods alone or in conjunction with iontophoresis have been suggested for applying tattoos. For example, in U.S. Pat. No. 6,565,532, Yuzhakov describes microneedles in conjunction with iontophoresis to apply tattoos. In U.S. Pat. No. 5,885,211, Eppstein discloses a microporation device for opening pores to apply a tattoo, which employs a heat-conducting element for applying the tattoo, rather than iontophoresis. Eppstein, in U.S. Pat. Nos. 6,527,716 and 6,692,456, discloses a microporation device for opening pores to deliver tattoo dye into the skin, which can employ iontophoresis. However, none of these discloses the information necessary to enable one of ordinary skill in the art to actually apply or remove a tattoo via iontophoresis alone, much less the information necessary to make a device or kit that can be employed by a layman to apply or remove a tattoo using iontophoresis alone.

There is thus a widely recognized need for, and it would be highly advantageous to have a body decoration system and method of use thereof which is devoid of the above limitations. It is therefore desirable to have the benefit of body art, or semi-permanent skin coloration, or semi-permanent make-up, or a tattoo, or skin decoration, that would be temporary, yet lasting more that a few days. It is further desirable to have such a body decoration system, which does not involve painful procedures and the use of needles. Furthermore, it is desirable to allow the wearer to perform such skin decoration individually, at the home setting, without the need to use costly professional salons. Still further it is desirable to have a body decoration system, which uses non-toxic dyes. Finally, it is desirable to have such a system, which has low cost. Preferably, such a system should be disposable.

### SUMMARY OF THE INVENTION

Embodiments of the present invention include a kit for applying a body decoration to a subject. The kit may include a color formulation, means for applying the color formulation to a body area of a subject, and an electrically powered patch that promotes penetration of the color formulation into the body area.

Embodiments of the present invention also include a patch device to promote penetration of a color formulation into a body area of a subject, and a method for applying a body decoration to a subject.

These embodiments provide numerous advantages, including body decoration that may be temporary, yet lasting for more than a few days, body decoration that does not involve painful procedures and the use of needles, and body decoration that allows the wearer to perform the application at home and at low cost, with a kit that may be disposable. Further aspects of the invention described herein are set forth in the appended claims.

Embodiments of the present invention also provide a kit, a patch device, and a method for removing the body decoration from the subject.

The term ‘body decoration’ as used herein includes, but is not limited to any form of graphic decoration or marking of any suitable body part and/or any form of skin coloration, including temporary and semi-permanent and permanent body decoration. The term includes tattoos and make-up and any other suitable artistic or graphical or decorative body marking. The term also includes body decoration resulting from use of any type of ink/pigment or colorant or other marking means.

### BRIEF DESCRIPTION OF DRAWINGS

FIGS. 1A and 1B are an embodiment of an iontophoretic patch according to the present invention;

FIG. 1C is an alternative embodiment of an iontophoretic patch according to the present invention;

FIG. 2 is a flowchart of a method according to an embodiment of the present invention;

FIGS. 3 and 4 illustrate a method according to embodiments of the present invention;

FIG. 5 illustrates exemplary body decorations that may be applied according to embodiments of the present invention; and

FIGS. 6-14 are examples of body decorations resulting from application of embodiments of the present invention.

### DETAILED DESCRIPTION

Embodiments of the present invention provide a kit for application of a decorative form to a body area of a subject. The kit may include a color formulation, including dyes and/or pigments, suitable for decorating the body, a means for applying the formulation to the desired body area, and an electrically powered patch coupled to the means for applying. In one embodiment, the formulation may be applied to the desired body area using a stencil that is not attached to the patch. The color formulation may be contained in a conductive composition on an electrode included in the patch and applied to the desired body area when the electrode is placed over the stencil, or the color formulation may be contained in an independent container and applied directly to the stencil from that container. In another embodiment, the formulation may be applied to the desired body area through a transferable sheet that is also not attached to the patch. In yet another embodiment, the formulation may be applied to the desired body area by making a free-hand drawing on the desired body area with the formulation. In yet still another embodiment, the formulation may be applied to the conductive layer by making a free-hand drawing on the conductive layer, and then applied to the body area of a subject using the patch.

The patch included in the kit may comprise an electrochemical cell having at least two electrodes positioned on one side of the patch to form electrical contact with the body area of the subject and a conductive substance to provide a conductive interface between the patch and body area, wherein the electric current is introduced into the body area to promote the penetration of the formulation into the body area. The technique used may be iontophoresis. In this application, the term “iontophoresis” may comprise any method of electrical delivery of substances to the body of a subject, including iontophoresis, electroosmosis and electroporation.

Embodiments of the present invention also provide a fully integrated, “all-in-one” patch device for application of a decorative form to a body area of a subject. The fully integrated patch device includes the elements of a kit (i.e., a color formulation, a means for applying, and a patch), but those elements are all integrated into the patch. There are many different embodiments of the fully integrated patch device. For example, in one embodiment, the patch device may have a stencil attached to an active electrode on the patch, with the color formulation contained on that electrode. In another embodiment, the patch device may include an electrode cut into the shape of the decorative form, with the color formulation contained in a conductive composition cut into that same shape and attached to that same active electrode, or with the color formulation (without a conductive composition) contained in a separate chamber attached to the electrode.

The fully integrated patch device may include at least one electrochemical cell having at least two electrodes positioned on one side of the patch to form electrical contact with a body area of a subject, a conductive substance attached to the electrodes which provides a conductive interface between the patch and the body area, a colored formulation attached to the conductive substance and which includes dyes and/or pigments suitable for decorating the body and an attached means for applying the formulation to the desired body area (such as the stencil), wherein the patch introduces electric current into the body area to promote the penetration of the formulation into the body area. Preferably the patch components are attached to a patch substrate.

Embodiments of the present invention also provide a method for applying a decorative form to a body area of a subject. The method may include contacting a body area with the means for applying, applying a color formulation to the body area with the means for applying, and promoting penetration of the color formulation into the body area with an electrically powered patch in contact with the formulation via iontophoresis.

In one embodiment of the method, the kit is used to apply a decorative form to the body area of a subject. In this embodiment, the subject may adhere a separate stencil (i.e., a stencil that is not attached to the patch) of the desired decorative form to the desired body area and then apply the color formulation as outlined by the stencil. The color formulation can either be contained in a conductive composition on the electrode (and applied by covering the stencil with the patch), or contained in an independent container and applied directly to the stencil from that container. As an alternative, the subject may adhere a transferable sheet to the desired body area and then apply water to the sheet to cause a color formulation in the sheet to apply to the body area in the desired decorative form, remove the sheet, and then apply the patch to the decorative form previously applied to the body area with water to promote the penetration of the color formulation in the decorative form into the body area. The subject may apply the patch directly to the sheet to promote the penetration of a color formulation in the sheet into the body area without a wetting step. As another alternative, the subject may make a free-hand drawing of the desired decorative form with the color formulation, and then apply the patch to the free-hand drawing to promote the penetration of the color formulation in the drawing into the body area. As yet another alternative, the subject may use the color formulation to make a free-hand drawing of the decorative form on a conductive layer (e.g., a hydrogel), and then apply that decorative form to a body area of a subject using the patch.

In another embodiment of the method, the fully integrated patch device is used to apply a decorative form to the body area of a subject. In this embodiment, the subject may contact and/or adhere the patch device, which includes an attached stencil/template of the desired form, to the desired body area. The color formulation is preferably contained with the conductive substance, which is attached to the active electrode. As an alternative, the patch device may include an electrode cut into the shape of the decorative form, with the color formulation contained in a conductive composition cut into that same shape and attached to that same active electrode, or with the color formulation (without a conductive composition) contained in a separate chamber attached to the electrode.

The method may optionally include applying a body area treatment prior (pre-treatment) to the decorative template, such as, but not limited to, cleaning the body area for the decoration and/or peeling treatment to increase penetration of the color formulation. The method may also optionally include applying a sealant after the decoration (post-treatment) to improve decoration preservation. In embodiments of the present invention, body decoration may be applied to the skin, nails, teeth, hair, lips, and any other body area suitable for having body decorations.

Embodiments of the present invention advantageously ensure durable, attractive body decoration without the drawbacks of existing tattooing approaches. In particular, embodiments of the present invention provide improved graphic decorative forms, durable but temporary forms, color formulations that are safe to the body, non-invasive application, and disposable at-home application.

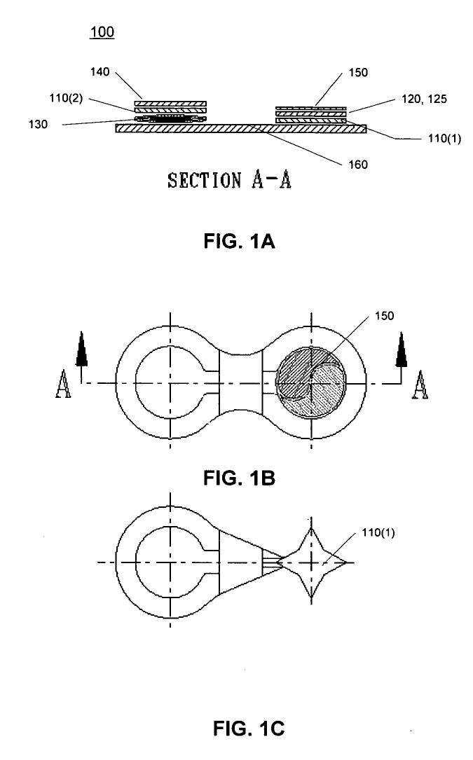

FIG. 1A depicts an embodiment of a fully integrated iontophoretic patch device for body decoration according to the present invention. FIG. 1A shows a cross-sectional view of the patch of FIG. 1B along line A-A. In this embodiment, patch 100 may comprise first electrode 110(1), identified as “cathode,” second electrode 110(2), identified as “anode,” and electrochemical cell 130 as the power supply of patch 100. Patch 100 may also comprise conductive layer 140 to provide an interfacing layer between patch 100 and a body area of a subject. Patch 100 may also comprise conductive layer 120, which in FIG. 1A includes color formulation125, and which is preferably a conductive composition to provide dyes and/or pigments to a desired body area for the decoration. Optionally, patch 100 also comprises a decorative template 150 to provide the decorative form to be made on the body area. In a preferred embodiment as shown in FIG 1A, the electrodes, conductive layer, color formulation, and electrochemical cell may be supported on substrate 160. Electrochemical cell 130 may optionally be disposed on substrate 160, electrode 110(2) disposed on electrochemical cell 130, and conductive layer 140 disposed on electrode 110(2). Electrode 110(1) may be disposed on substrate 160 in spaced relation to electrochemical cell 130 and electrode 110(2) to define a gap between the two electrodes. Conductive layer 120 including color formulation 125 may be disposed on electrode 110(1) and decorative template 150 disposed on conductive layer 120 including color formulation 125. In an alternative embodiment, patch 100 does not include conductive layer 120 (or conductive layer 140). In this alternative embodiment color formulation 125 is accommodated in a chamber (not shown in figure) attached to electrode 110(1). Color formulation is then applied without conductive fluid onto decorative template 150 to form desired body decoration.

FIG. 1B is a plan view of the embodiment depicted in FIG. 1A. In FIG. 1B, the two groups of stacked components may be separately spaced on substrate 160 as described previously with conductive layer 140 on top of one stack and decorative template 150 on top of the other stack. Accordingly, application of the patch in this embodiment may apply the decoration (which in this non-limiting example is a circle with an S-shaped curve inside it) as decorative template 150 in FIG. 1B to the body area of the subject.

As noted, the embodiment depicted in FIGS. 1A and 1B is a fully integrated patch device. An embodiment of the patch used as part of a kit in accordance with the present invention is similar to the embodiment of the fully integrated patch device depicted in FIGS. 1A and 1B. However, in this alternative embodiment, the patch (when it is part of a kit) may be used with a separate decorative template (rather than having an integrated decorative template 150). Or, the conductive layer 120 of the patch (when it is part of a kit) does not include a color formulation, with the color formulation instead being supplied by other means, such as through a color formulation contained in a separate container that is applied to a decorative template, a color formulation that is part of a transferable sheet, or a color formulation that is applied directly to the body area as a free-hand drawing is made.

Certain features of the patch of the present invention that are the same regardless of whether the patch is a fully integrated patch device or a patch that is part of a kit will now be described. Preferably the patch, including its components, is thin and flexible, to suit the contour of a body area of a subject. The patch may be electrically powered. The patch may be any size, color and shape suitable for application to a desired body area. The thickness of the patch is preferably up to 10 mm to ensure flexibility, but may be thicker, depending on the application. The patch is preferably disposable, but may be reusable. The patch is stable to a wide range of temperatures and humidity.

The power supply of the patch is optionally any suitable power supply. Preferably, the power supply is at least one electrochemical cell. According to a preferred embodiment of the present invention, the electrochemical cell of the patch may be a thin, flexible and disposable electrochemical cell. The thickness of the power cell can be up to 4 mm, more preferably up to 2 mm and most preferably up to 1 mm. In the presently preferred embodiment, the electrochemical cell may include a positive pole layer, a negative pole layer, and an electrolyte layer interposed therebetween. An example of a suitable thin and flexible electrochemical cell is described, for example, in U.S. Pat. Nos. 5,652,043, 5,897,522 and 5,811,204, which are incorporated herein by reference. Briefly, the electrochemical cell described in the above-identified U.S. Patents is an open liquid state, electrochemical cell, which can be used as a primary or rechargeable power source for various miniaturized and portable electrically powered devices of compact design. The cell may comprise a first layer of insoluble negative pole, a second layer of insoluble positive pole, and a third layer of aqueous electrolyte being disposed between the first and second layers and including (a) a deliquescent material for keeping the open cell wet at all times; (b) an electroactive soluble material for obtaining required ionic conductivity; and (c) a water-soluble polymer for obtaining a required viscosity for adhering the first and second layers to the third layer.

Optionally, the power supply in the patch is a single electrochemical cell. However, the power supply need not be limited to one cell, but may include a plurality of connected electrochemical cells, a plurality of batteries, and/or electronics configured to increase, control, and change phase of the supplied electric current and wherein the power supply is thin and flexible. The electrochemical cell in the patch preferably provides electrical potential (voltage) to the desired body area of the subject in the range between about 0.5V and about 12V and more preferably in the range between about 1V and about 9V. In a preferred embodiment, the electrical potential may be adjusted to satisfy at least one of the following three criteria.

First, the patch voltage may be adjusted to enable an iontophoretic delivery of the dyes and pigments into the body area. For that purpose, voltage may be adjusted to provide an electric current of between about 0.02 mAmp/cm² and about 1 mAmp/cm² to the body area. A preferred electric current may be between about 0.02 mAmp/cm² to about 0.5 mAmp/cm². The electric current may be applied as direct current, pulse current, alternating current or in any other such manner suitable for providing the desired current for applying the decoration.

Second, the patch voltage may be adjusted to minimize the penetration of the dyes and pigments through the body, and to maximize the amount into the desired body area. Thus, using an in-vitro skin penetration model and applying the selected voltage, the amount of dyes and pigments found in the body may be higher than the respective amount found in the receiving compartment of a modified Franz cell, as described later in regard to the experiments. Thus, in a preferred embodiment, the electrochemical cell voltage may be in the range between about 0.5V and about 12V; and in a more preferred embodiment, the voltage in the range between about 1V and about 9V; and in a still more preferred embodiment, the voltage in the range between about 1V and about 4.5V.

Third, the patch voltage may be adjusted to minimize body area irritation, which may result from excessive electric current, passing into and through the body. Thus, in a preferred embodiment, the electrochemical cell voltage may be in the range between about 0.5V and about 12V; and in a more preferred embodiment, the voltage in the range between about 1V and about 9V; and in a still more preferred embodiment, the voltage in the range between about 1V and about 4.5V.

The power supply may optionally be located in any suitable position on the patch. In the embodiment shown in FIG. 1A, the power supply is disposed between electrode 110(2) and substrate 160. This configuration may be preferable when electrode 110(2) does not supply electric current to the body area. Such may avoid electrical interference between the power supply and electrode. In addition, this stacked configuration may minimize wasted space on the patch. Alternatively, when electrode 110(2) does supply electric current to the body area, electrode 110(2) may be disposed on substrate 160 and the power supply may be disposed on substrate 160 in another position so as to maintain a spaced relation between the power supply and each of the electrodes.

A power supply to the patch may provide a duty cycle and pulse partition rate of between about 10% and about 90%. The frequency of the power supply may preferably be from about 1 Hz to about 100 Hz. The power supply may provide voltage in a preferable range of from about 0.5V to about 100V to the patch.

Cathode and anode electrodes 110(1) and 110(2) are preferably composed of a conductive material. In a preferred embodiment, at least one of the electrodes may comprise silver metal. In a further preferred embodiment, at least one electrode may comprise both silver and silver chloride. Yet, in other preferred embodiments, at least one of the electrodes may comprise carbon or zinc. Alternatively, at least one of the electrodes may comprise graphite or platinum. Any other conductive element or compound, including metal and non-metal materials, can be used as electrode materials. The electrodes may be either provided as thin sheets, linked to the power source, or printed onto a substrate in spaced relation to each other to define a gap therebetween. The electrode area can be continuous, or formed as a drawing, in any shape, to provide a decorative form. Optionally, patch can include a plurality of anodes and a plurality of cathodes. Such a multi-electrode patch facilitates providing simultaneously a plurality of body decorations in different body areas.

In the embodiment shown in FIG. 1A, cathode electrode 110(1) is active. However, either anode electrode 110(2), cathode electrode 110(1), or both electrodes may be active for applying body decorations. In one embodiment, the cathode may apply a negatively charged dye. In another embodiment, the anode may apply a positively charged dye. In another alternate embodiment, both the anode and cathode may be used to produce a body decoration, such that multiple body decorations may be applied concurrently. Alternatively, a neutral dye may be used in combination with electroosmosis or a chemical ionization enhancer. Thus, at least the features of the patch of the present invention described above are the same regardless of whether the patch is a fully integrated patch device, or a patch included as part of a kit.

In the embodiment of the fully integrated patch device shown in FIG. 1A, conductive layers 140 and 120 may each comprise a conductive hydrogel. Decorative template 150, e.g., a stencil, may be an insulating material interposed between patch 100 and the body area to limit the application of color formulation 125 to the body area exposed by the template and to limit electric current from electrode 110(1) to the body area where color formulation 125 is applied. Any suitable insulating material may be used, but polyester may be preferred. A plurality of decorative templates may be interchangeably used with the patch. Preferably, template 150 includes a cut out of the desired tattoo design. The use of template 150 advantageously produces the decoration without wasting electric current. Optionally, decorative template 150 may be omitted entirely. Or, the decorative template may be included as part of a kit that also includes a patch.

In an alternate embodiment, conductive layer 140 may include a conductive hydrogel and a color formulation. In such an embodiment, a decorative template of insulating material in the shape of the decoration may be interposed between the conductive layer and the body area to limit the application of the color formulation and electric current to the desired body area. In such an embodiment, both electrodes may be active to provide body decorations respectively, through conductive layer 140 and through conductive layer 120.

Alternatively, the conductive layer may be a conductive adhesive, such as but not limited to hydrogel, which is cut or made into a desired decorative form and comprising a conductive material with a color formulation. In such an embodiment, the adhesive may contact the body area such that the color formulation is applied in the shape of the adhesive and current is limited to the body area in contact with the adhesive. In this embodiment, it is preferred that the electrode in contact with the conductive layer also be cut or made into the shape of the desired decorative form.

In another embodiment, the conductive layer may be a hydrogel preprinted with the desired decorative form using the color formulation.

In the embodiment of FIGS. 1A and 1B, patch 100 is configured to attach to the body area by conductive layer 140. In alternate embodiments, the patch may be attached to the body area by other attachment means such as, but not limited to the frame of the substrate and/or attachment means on the substrate. Such attachment means may include adhesive strips, for example.

It is to be understood that the configuration as shown in FIGS. 1A and 1B is for illustration and is not intended to be limited to that shown. The components of the patch may be arranged on the substrate or any other base layer in any suitable manner, to facilitate body decoration according to embodiments of the present invention. Alternatively, the color formulation, the cathode and anode conductive layers, and the decorative template may be detachable from the patch and reattached as needed.

In summary, the patch of the present invention can be a fully integrated patch including the conductive layer, color formulation and decorative template, or a partially integrated patch including just the conductive layer and color formulation without the decorative template. In an alternative embodiment, the patch is not a fully or partially integrated patch, but instead is part of a kit including separate components, such as a separate conductive layer containing a color formulation or a separate decorative template.

In a preferred embodiment of the present invention the body decoration patch is a printed patch, wherein at least one of the components is printed. Preferably, the body decoration patch is a fully printed patch, wherein the battery, electrodes, conductive layer, body decoration color/dye and design template are printed using a suitable printing technique.

FIG. 1C is an alternate embodiment of a fully integrated iontophoretic patch for body decoration according to the present invention. In this alternate embodiment, electrode 110(1) may be formed in the shape of a decorative form to be applied to the desired body area. In FIG. 1C, electrode 110(1) is formed in the shape of a star. In this embodiment, decorative template 150 may be omitted, and conductive layer 120 including color formulation 125, is also in the shape of the decorative form, i.e., the star, may be disposed between electrode 110(1) and the body area.

In the context of the present invention the term ‘color formulation’ includes any type of ink as defined herein. The color formulation can optionally include any suitable additive as described herein including colorants, bases, solvents, buffers, resin, adhesives, humectants, flavor and fragrance. The optional use of a fragrance facilitates the production of a body decoration with a distinctive smell. The optional use of a flavor facilitates the production of a body decoration with a distinctive taste. The ink can be dry or wet. In the context of the present invention, the term “ink” relates to a formulation or paste or powder comprising dyes and/or pigments, to be applied in conjunction with the patch. Generally, many variants of inks can be used for body decoration according to embodiments of the present invention. Because ink formulas may have various chemical compositions and include various colorants and additives, there are many possible combinations when ink formulas are composed. Of the three common chemical bases used to formulate inks, water and petrochemical solvents are most common. The third and most atypical base used is oil, found mainly in wide-format commercial printers.

The colorant, or the substance used to give color to the ink, may be dye and/or pigment. Dye, comprising small molecules, blends with the water-based solution. A water-dye-based ink tints or stains paper on a molecular level. Because the dye is composed of single molecules, the dye may lie flatter on a paper surface, reflecting light more evenly and appearing more vivid. However, the smaller molecular structure of the dye-based ink may also allow the ink to be damaged by UV light more rapidly that pigmented inks.

Pigment comprises larger molecules than dye; therefore the reflection of light received from a pigmented print may not appear as vibrant due to the scattering of the reflected light. The larger molecules may allow pigmented ink's print to last substantially longer than a dye-based ink's print.

Hybrid ink including both dyes and pigments can also be used.

In addition to the chemical base and colorant, inks also contain additives. Additives may include buffering agents for control of the inks' pH levels, resin for resilience, adhesive materials and humectants for the prevention of evaporation.

Thus, many possible dyes and pigments can be selected for use in body decoration according to the present invention. Dyes and pigments may be selected from the list provided in an FDA document, entitled “Summary of Color Additives Listed for Use in the United States in Foods, Drugs, Cosmetics, and Medical Devices,” which is published on the FDA internet site, http://www.cfsan.fda.gov/˜dms/opa-col2.html. The detailed lists can also be found in Title 21 of the Code of Federal Regulations Parts 73 and 74. Dyes and pigments for use for body decoration according to the present invention are preferably selected according to the following criteria.

The color (or colors) of the dye or pigment to be used may be selected according to decorative considerations or other considerations. Such colors may include single colors, combinations of colors, mixtures of colors, fluorescent colors, glitter, metallic colors, melanin, skin colors, and any other desirable color suitable for body decoration. A mix of dyes and pigments can be used to attain specific tones, which are not available by using single dyes and pigments. In order to provide sophisticated body decoration, one can apply different colors, combined together in one unit or applied step-wise, to attain a colorful picture.

Dyes and pigments, which are water soluble, may be preferred to such that are not water-soluble. For the purpose of the present invention, the term “water soluble pigment” stands for a pigment that can dissolve in water in concentration of at least about 0.01% on a weight-by-weight basis. Water-soluble dyes and pigments can be in an ionic and non-ionic form, and if ionic, they may be singly or multiply charged. They may be positively charged (cations) or negatively charged (anions).

Thus, the decoration can be designed as either monochrome or multi-color decoration, can have different sizes, and can include artwork, whether original or licensed. In one embodiment, different colors may be used to produce a three-dimensional effect in the decoration.

Optionally, dry inks can be used or wet inks. Preferably, dry inks are activated by a suitable liquid or semi-liquid, such as for example, but not limited to water.

In a preferred embodiment, dyes and pigments may be such that they adhere to body outer tissue, in order to provide a prolonged retention of the decoration in the body. The adherence may be based on either chemical bonding to tissue materials, such as proteins, glycoproteins, glycolipids, polysccharides and the like; by physical forces of adherence; by binding to keratin filaments; or by solubilization in an intercellular space or in a body cell.

As noted, embodiments of the present invention may be directed to the provision of a kit, comprising a color formulation (i.e., any type of ink), including dyes and/or pigments, suitable for decorating the body, a means for applying the formulation to the desired body area, and an electrically powered patch coupled to the means for applying. According to one embodiment, the kit can be used sequentially, whereby the color formulation is first applied, using a means of application, as will be described later. Yet, according to another embodiment of the same invention, the color formulation may be located on the patch, which is applied on the body area.

The advantage of iontophoretic delivery of dyes and/or pigments is in the fact that the electric current, which mobilizes such substances, may be passed in a vertical direction, thus delivering the dyes and/or pigments in a focused fashion to their desirable location, according to the designated graphic design.

As stated, in one embodiment of the invention, the delivery of the dye and/or pigment preferably occurs by a process of iontophoresis, electroosmosis and/or electroporation (all of which are encompassed under the term “iontophoresis” according to the present invention). Iontophoresis refers to the movement of ions caused by the application of an electrical potential. Electroosmosis refers to the convective movement of solvent that occurs through a charged “pore”, in response to the preferential passage of counter-ions when the electric field is applied. It may be used as a means to augment the anodic delivery of (in particular) large, positively charged compounds, and to promote the intradermal and transdermal penetration of uncharged, yet polar, molecules. Electroporation refers to the movement of charged colloidal particles or macromolecules caused by the application of an electrical field. The electric current caused by the electric potential between the two electrode (anode and cathode) serves to deliver the dye and/or pigment from the superficial layer of the body area into the adjacent body tissue.

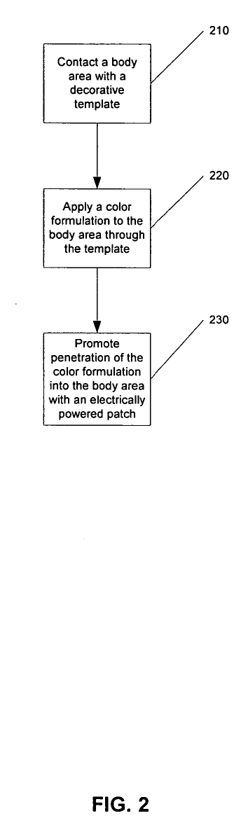

FIG. 2 is a flowchart of a method according to embodiments of the present invention. The flowchart applies to a method using a fully integrated patch, or to a method using a kit including a patch. First, a subject may contact (210) a body area with a decorative template (which may or may not be part of a patch). The subject may then apply (220) a color formulation through the template onto the body area. Exemplary methods of applying the color formulation are described below. Next, the subject may promote (230) penetration of the color formulation into the body area through the use of an electrically powered patch. The patch may be used on the body area for a period of 0-10 hours, and preferably about 2 hours.

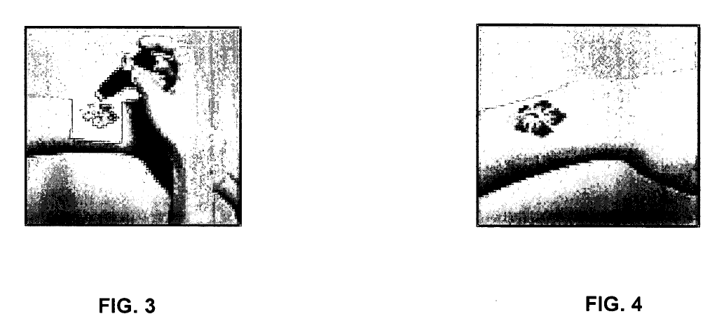

FIGS. 3-4 illustrate one embodiment of application of body decoration in accordance with the present invention. FIGS. 3-4 illustrate the use of a kit to practice a method of the present invention. In FIG. 3, the decorative template is a stencil of a desired decorative form that is not attached to a patch. The stencil has a backing sheet that preserves an adhesive on one side of the stencil. When the backing sheet is removed, the subject may adhere the stencil to the desired body area. The subject may then apply the color formulation to the body area through the stencil. The subject may apply the color formulation either by hand, as in FIG. 3, or with an electrically or mechanically powered color delivery device. The subject may apply the electrically powered patch to the body decoration to promote the penetration of the color formulation into the body area by iontophoresis. The subject may then remove the stencil. The result is a body decoration in the shape of the stencil, as shown in FIG. 4.

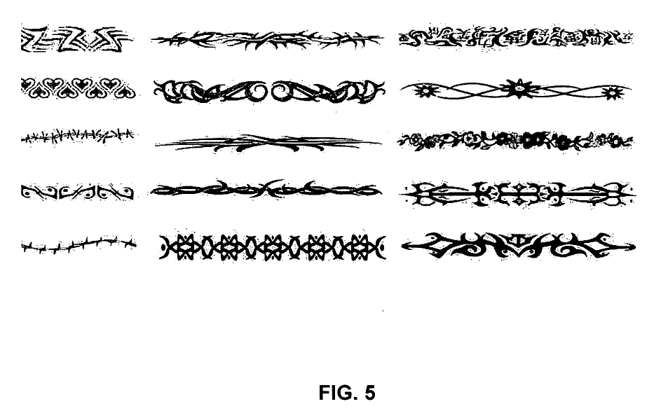

FIG. 5 shows exemplary body decorative forms that may be applied as body decorations. It is to be understood that these forms are for illustration only and that any desired form may be used.

In embodiments of the present invention, a variety of body decorations may be applied. For example, decorations with different textures may be applied as determined by the color formulation applied and/or the decorative template used and the electrode used. An electrode that facilitates providing different currents per area may provide such a textured body decoration. Any one of a smooth, rough, furry, granulated, etc. body decoration may be produced.

The decorative template may be made from any material suitably sturdy to maintain the desired decorative form, sufficiently flexible to adhere to a desired body area, and insulating to limit application of electric current to the body area to which the color formulation is to be applied. A preferred material may be polyester. However, other materials having the above mentioned properties may be used.

In an embodiment of the present invention, the method may also include the subject pretreating the body area prior to contacting the body area with the decorative template. The pre-treatment may include light peeling, particularly when the body area is the skin, by any suitable method, such as physical and chemical peeling, iontophoretic peeling, or application of a scrub composition. Pre-treatment may be preferably done with a lactic acid solution in a range of from about 2% to about 20% solution. More preferably, pre-treatment may be done with a 10% lactic acid solution. Pre-treatment may also include cleaning the body area. Pre-treatment may also include applying an electric current to the body area using an iontophoretic patch of the present invention that does not include a color formulation. Pre-treatment facilitates increased penetration of the tattoo ink resulting in an improved tattoo.

The method may also include the subject post-treating the body area after the patch has promoted penetration of the color formulation into the body area. A problem associated with the tattoo is wearing away of the color by exposure to water from washing. The post-treatment may include applying a sealant to preserve the body decoration for a longer period of time and to prevent water penetration. Post-treatment may be preferably done with a spray clear plaster or any suitable long lasting colorless, water repellant composition.

In embodiments of the present invention, the subject may control the color and life span of the decoration. The subject may use either permanent or temporary color formulations, may chose the colors to be used, the design to be applied, determine whether to apply a sealant, or adjust the penetration depth of the ink into the body area, for example. The life span of the decoration may in part be a function of the penetration depth, which may in part be a function of the length of time that the patch is used to promote penetration. The patch may be used for 0-10 hours, and preferably for about 2 hours.

Accordingly, the present invention provides a device, kit and method for applying a body decoration (such as a tattoo), which facilitate providing a body decoration lasting different durations of time. The present invention can result in temporary body decorations (tattoos), lasting from several hours to several months. Alternatively, the present invention can also provide permanent body decorations (tattoos). Preferably the duration of the tattoo is up to about 1 month.

In an alternate embodiment of using a kit for application of body decoration in accordance with the present invention, the subject may contact the desired body area with a transferable sheet as the decorative template. The transferable sheet may be coated with the color formulation in the desired decorative form on one side of the sheet. The sheet may have a protective cover to adhere the coating to the sheet until application. The subject may peel off the protective cover and place the transferable sheet onto the body area with the coated side in contact with the body area. The subject may then apply a wet cloth for a predetermined time period to the sheet to wet the sheet thoroughly. A preferable time period may be about 30 seconds. The subject may then remove the wet cloth and apply the patch to the sheet or to the transferred decorative form on the body area to enhance the formulation application via iontophoresis. The subject may then remove the sheet from the body area. The result is a decorative form transferred from the sheet onto the body area. The subject may optionally perform a pre-treatment and/or post-treatment.

In another alternate embodiment for application of body decoration in accordance with the present invention, the subject may contact the desired body area with a sheet pre-fabricated, pre-coated, or pre-impregnated with conductive elements and shaped into a decorative form as the decorative template. The conductive sheet may be attached to the patch such that there is a seemingly one-step application (which is an embodiment of the fully integrated patch device), but the sheet may also be separate from the patch (which is an embodiment of the kit including a patch). In this embodiment, the patch may apply an electric current that is transmitted into the body area through the conductive elements to enhance the transfer of the coating from the sheet onto the desired body area. In one embodiment, the sheet may be detachable from the patch such that different sheets may be reused with the patch.

The conductive elements' resistance may be variable to adjust and control the penetration depth of the decoration into the body area. The sheet of this embodiment may provide a body decoration with a three-dimensional aspect. In this manner, conductive elements with different resistances may be used on different areas on the sheet, with the result that a dye and/or pigment on one area of the sheet penetrates farther into the body area than a dye and/or pigment on another area of the sheet.

In another embodiment of the present invention, ink may be applied on the patch prior to usage for body decoration. The ink, or pluralities of inks, in different colors may be placed on a patch surface, according to a desirable graphic design. The design and application of ink on the patch surface can be carried out by computerized or non-computerized printing, as well as manual application, by free-hand drawing, using templates, silk screen print, stencil, and any other method of application. The preferred patch is the computerized or non-computerized printed patch.

In a further embodiment, as shown in FIG. 1C, for example, the patch electrode may be shaped in the desirable graphic design and the ink, or a plurality of inks, in different colors, may be placed on a patch surface, according to the same design, or otherwise. Upon application of the patch on the body area, dyes and/or pigments may be delivered into the body area specifically according to the designated graphic design.

Likewise, inks can be applied using freehand drawing, silk print, lithography and stamp-like applicators, as well as any alternative way of ink application. In this manner, inks can be applied to the body area first and then driven into the body area by the patch. Alternatively, inks can be applied to the patch first and then the ink-applied patch can be applied to the body area to drive the inks into the body area.

An in-vitro skin penetration study was conducted to determine which combination of parameters results in the maximum amount of active substance, e.g., color formulation, being delivered into the skin, i.e., the dermis and epidermis, but not through the skin, i.e., beneath the epidermis. Such a study enabled the present inventors to design an effective iontophoretic patch for body decoration. According to embodiments of the present invention, following the application of the ink and the patch, the dye and/or pigment are found ideally in the skin, i.e., the dermis and epidermis, with minimal penetration through the skin.

The in-vitro skin penetration study generally included the following test procedure. Decoration ink, as in embodiments of the present invention, was applied on a piece of swine ear skin. The swine skin was excised to full thickness (epidermis and dermis) to an approximate depth of 500-1000 μm. The skin was removed from the ear within a few hours of sacrifice and was either used immediately or stored frozen for a period of no longer than 2-3 weeks. The ink-applied swine skin was thereafter placed in vertical diffusion cells, in which the skin membrane separated the physically and electrically isolated anode and cathode chambers from the receptor phase. See, e.g., P. Glikfeld, et al., “A new system for in vitro studies of iontophoresis,” Pharma. Res. 5: 443-446 (1988). Alternatively, the swine skin may be placed in modified side-by-side cells of a newer design. The vertical cells' receptor compartment contained physiologically buffered saline (PBS) at pH 7. Then, two electrodes, i.e., anode and cathode, were placed on the swine skin over the ink. The exposed area of skin in each electrode chamber was 0.64-0.67 cm².

In each experiment of the study, preparation with electric current (“Active”) and without electric current (“Passive”) were tested. Exposure period was typically 20-30 minutes, but can be altered according to the designated time of patch application. Six replicates were performed per experiment. Prior to each experiment, the viability and integrity of the swine skin barrier function was checked via a measurement of transepidermal water loss (TEWL). Perturbation of the skin barrier, either by physical disruption, chemical attack, or because of disease, could severely compromise the role of the stratum corneum (“SC”).

At the end of the exposure period, the entire receptor compartment was drained and the solution reserved for subsequent analysis of the dye(s) and/or pigment(s). The swine skin was then removed from the receptor compartment, and the skin's surfaces carefully cleaned and dried. Subsequently, the stratum corneum (“SC”) beneath the cathode chamber was removed by 15 adhesive tape strippings (“TS”). The depth of penetration was measured by noting the number of tape strippings required to remove the ink. See Table 1. The ink was extracted from the respective tapes and assayed to yield a total uptake into the SC. The remaining SC-stripped skin from beneath the cathode was then appropriately treated so as to recover the ink, which had crossed the SC barrier, during iontophoresis, and reached into the underlying epidermis/upper dermis. The ink was determined quantitatively using customary analytical procedures (e.g., HPLC, UV-Vis spectrum, GC, radiolabeled detection, etc.), as applicable.

The in-vitro skin penetration study was conducted using an in-vitro modified Franz Cell System, equipped with the necessary components to exert micro-powered electric current, which can simulate the expected effect of the iontophoretic patch of the present invention.

This study may be performed on viable skin tissue of many species, including human, pig, mouse, rabbit, and rat. As such, in-vivo human studies were conducted to demonstrate the ability of embodiments of the present invention to tattoo human skin even when using low electric currents.

In the first part of the in-vitro skin penetration study, the color application included the use of dyes D&C Green no. 6, FD&C Blue no. 1, FD&C Yellow no. 6, and FD&C Red no. 40. All chemicals used were analytical grade, obtained from standard supply houses (Sigma, VWR, etc.). The dyes were dissolve in ddw and the solution conductivity was measured using a dedicated apparatus. The target conductivity was defined as a minimum of 1 mAmp/cm³ current at 3 volts. If such conductivity was not attained, the dye concentration and/or the voltage were increased. See Table 1 for the solubility and conductivity of the dyes.

TABLE 1

| | Molecular | Concentration | | | | |
|---|---|---|---|---|---|---|
| Dye | weight (g/mol) | (In water) | Solubility | Voltage | Current | Toxicity |
| Blue no. 1 | 792 | 2 mg/ml | Soluble | 3 V | None | 5.5 gm/kg |
| | | 6 mg/ml | Soluble | 3 V | ˜0.3 mA | SC in mice |
| | | | | 3.92 V | 1 mA | |
| | | 10 mg/ml | Soluble | 3 V | ˜0.4 mA | |
| | | (1 gm/kg in mice) | | 3.69 V | 1 mA | |
| | | 20 mg/ml | Soluble | 3 V | ˜0.4 mA | |
| | | | | 3.69 V | 1 mA | |
| Red no. 40 | 498 | 10 mg/ml | Soluble | 3 V | ˜0.3 mA | LD50 > 10 gm/kg, |
| | | | | 4.15 V | 1 mA | SC |
| | | 20 mg/ml | Soluble | 3 V | 1.1 mA | in rabbit |
| Yellow no. 6 | 452 | 20 mg/ml | Soluble | 3 V | 1.3 mA | LD50 = 4.6 gm/kg |
| | | (2 gm/kg in mice) | | | | IP in |
| | | | | | | mice |
| Green no. 6 | 418 | 10 mg/ml | Insoluble | | | LD50 = 0.25 gm/kg |
| | | 10 mg/ml + 30% | Insoluble | | | IP in mice |
| | | ethanol | | | | |
| | | 2.5 mg/ml + 30% | Insoluble | | | |
| | | ethanol | | | | |

In these experiments, the enhancing effect of iontophoresis and/or electroosmosis on skin penetration of different dyes was assessed. The assessment included evaluation of solubility and conductivity of the dyes followed by iontophoresis versus passive in-vitro skin penetration. The following properties were measured to determine the enhancing effect of iontophoretic induction on the dyes' skin penetration: uniformity of the dyeing by observation; and penetration depth by tape stripping.

The experiments performed in the first part of the study are described below. In these experiments, Hydroxyethyl cellulose with NaCl may comprise hydroxyethyl cellulose (Natrosol 250 HHBR, Hercules) at 1% wt., NaCl at 10% wt., and water at 98% wt. Hydroxyethyl cellulose without NaCl may comprise hydroxyethyl cellulose (Natrosol 250 HHBR, Hercules) at 1% wt. and water at 99% wt. 10% lactic acid of ph≅3.5-4 may comprise 90% lactic acid at 10% wt., NaoH 1M at 60% wt., and water at 30% wt. 5% lactic acid of ph≅3.5-4 may comprise 90% lactic acid at 5% wt., NaoH 1M at 30% wt., and water at 60% wt.

Experiment 1.1

The skin penetration of 10 mg/ml FD&C Blue no.1; 20 mg/ml FD&C Yellow no.6 and 20 mg/ml FD&C Red no. 40 in ddw was assayed. 1 ml of the dye solution was added to one side of the dual chamber and 1 ml of PBS to the other side. The receiving solution was PBS. Each dye solution was tested in triplicates with electrical current (active assay) and without electrical current (passive assay) for maximum 30 minutes. In the active experiments, Ag/AgCl wires were inserted into the chamber cells and the cathode/anode were attached to them depending on the dye charge. The required current was 500 μA/cm², thus the instrument was accordingly tuned to 335 μA (for 0.67 cm²).

Experiment 1.2

In the second assay the dyes concentration was increased and the current was unlimited. 1 ml of 100 mg/ml FD&C Blue no.1; 60 mg/ml FD&C Yellow no.6 and 60 mg/ml FD&C Red no. 40 in ddw were added to both donor chambers. Each dye solution was tested in duplicates, with and without electrical current for maximum 30 minutes.

Experiment 1.3

To improve the dyes penetration, the dyes were dissolved in ddw with 30% ethanol. 1 ml of 100 mg/ml FD&C Blue no.1; 25 mg/ml FD&C Yellow no.6 and 100 mg/ml FD&C Red no. 40 in ddw with 30% ethanol were added to both donor chambers. Each dye solution was tested in duplicates with and without electrical current for maximum 30 minutes (the current was unlimited).

Experiment 1.4

In this assay the experiment format was changed. Instead of dye solution we have used dry dye with conductive gel. 200 □l of 40 mg/ml FD&C Blue no.1 (dissolved in ethanol +20% ddw) was added to the donor chambers and was allowed to dry on the skin. 1-2 hours later, 1 ml of gel containing 39.2 g ddw, 0.4 g Hydroxyethyl cellulose (Natrosal) and 0.4 g NaCl was added to the chambers and the penetration of the dye into the skin was assayed with and without electrical current (the current was unlimited). The assay lasted 30 minutes and was done in duplicates. The dye penetration depth was determined by tape stripping.

Experiment 1.5

The skin penetration of the blue color mixture was tested under the same conditions as in the pervious assay. 200 μl of the blue color mixture (dissolved in 100% ethanol) was added to the donor chambers and was allowed to dry on the skin. 1-2 hours later, 1 ml of gel containing 39.2 g ddw and 0.4 g Hydroxyethyl cellulose (Natrosol; without NaCl) was added to the donor chambers and the penetration of the dye into the skin was assayed with and without electrical current (the current was unlimited). The assay lasted 30 minutes and was done in duplicates. The dye penetration depth was determined by tape stripping.

Experiment 1.6

To improve the skin penetration of FD&C Blue no.1, the Stratum Corneum was removed by repeated adhesive tape stripping (15 strips were removed). Other parameters were the same as described in the fourth assay.

Experiment 1.7

In this assay, prior to the experiment the Stratum Corneum lipid content was reduced by cleaning the skin with Acetone. The skin penetration of FD&C Blue no.1 was then tested under the same conditions as in the fourth assay.

Experiment 1.8

The skin penetration of 20 mg/ml FD&C Yellow no.6 and 20 mg/ml FD&C Red no. 40 was tested using skin that was cleaned with 70% ethanol. 200 □l of each dye (dissolved in ethanol +30% ddw) was added to the donor chambers and was allowed to dry on the skin. 1-2 hours later, 1 ml of gel containing 39.2 g ddw and 0.4 g Hydroxyethyl cellulose (Natrosol; without NaCl) was added to the donor chambers and the penetration of the dyes into the skin was assayed with and without electrical current (the current was unlimited). The assay lasted 30 minutes and was done in duplicates. The dye penetration depth was determined by tape stripping.

Experiment 1.9

The skin penetration of 20 mg/ml FD&C Yellow no.6 was tested under the same conditions as in the pervious assay except for the current that was limited to 335 mA (500 μA/cm²).

Experiment 1.10

The ability of the dyes to penetrate into the skin with limited current of 335 mA was assayed using skin without Stratum Corneum. Prior to the assay the Stratum Corneum was removed by repeated adhesive tape stripping (15 strips were removed). 200 μl of 40 mg/ml FD&C Blue no.1 (dissolved in ethanol +20% ddw), 20 mg/ml FD&C Yellow no.6 (dissolved in ethanol +30% ddw) and 20 mg/ml FD&C Red no. 40 (dissolved in ethanol +30% ddw) was added to the donor chambers and was allowed to dry on the skin. 1-2 hours later, 1 ml of gel containing 39.2 g ddw and 0.4 g Hydroxyethyl cellulose (Natrosal; without NaCl) was added to the donor chambers and the penetration of the dyes into the skin was assayed with and without electrical current (the current was limited to 335 mA). The assay lasted 30 minutes and was done in duplicates. The dye penetration depth was determined by tape stripping.

Experiment 1.11

To make the tattooing procedure more suitable for home consumers, in this assay we tested the ability of FD&C Red no. 40 to penetrate into skin that was cleaned with 10% lactic acid. 10% lactic acid, pH>3.5 is known to be used for home peeling (approved by FDA). Prior to the assay the skin was cleaned with 10% lactic acid pH=3.5-4. 200 μl of 20 mg/ml FD&C Red no. 40 (dissolved in ethanol +30% ddw) was added to the donor chambers and was allowed to dry on the skin. 1-2 hours later, 1 ml of gel containing 39.2 g ddw and 0.4 g Hydroxyethyl cellulose (Natrosal; without NaCl) was added to the donor chambers and the penetration of the dyes into the skin was assayed with and without electrical current (the current was limited to 335 mA). The assay lasted 30 minutes and was done in duplicates. The dye penetration depth was determined by tape stripping.

Experiment 1.12

In this assay the skin was cleaned with only 5% lactic acid to reduce possible skin damage that might occurred by cleaning the skin with 10% lactic acid. Other parameters were the same as in the previous assay using 40 mg/ml of FD&C Red no.40.

Experiment 1.13

To improve the dye uniformity in the present assay we have used hydrogel instead of conductive gel. Prior to the assay the Stratum Corneum was removed by repeated adhesive tape stripping (15 strips were removed). 200 □l of 20 mg/ml FD&C Red no. 40 (dissolved in ethanol +30% ddw) was added to the donor chambers and was allowed to dry on the skin. 1-2 hours later, pieces of hydrogel were placed in the donor chambers, on top of the dried dyes, and the penetration of the dyes into the skin was assayed with and without electrical current (the current was limited to 335 mA). In the active experiments, the cathode/anode were attached to the Ag/AgCl wires that were inserted into the chamber cells, touching the hydrogel. The assay lasted 30 minutes and was done in duplicates. The dye penetration depth was determined by tape stripping. Seven minutes after the beginning of the assay the charge rose to 8V to achieve the desirable current of 0.335 mA. To reduce the voltage, it was limited to 3V from this minute and until the end of the assay.

Table 2 shows the results of Experiments 1.1-1.13. According to the results, dye penetration into the skin with dyeing uniformity can be achieve by using, but not limited to, the following parameters: removing the stratum corneum by using tap strips or cleaning the skin with 10% lactic acid (pH>3.5); using dried color with conductive gel, preferably hydrogel; and placing the dyes udner the correct electrode, depending on the dye charge. According to the results, the dye penetration into the skin occurred when dried color with conductive gel or hydrogel were used, as in Experiments 1.4-1.13. Hydrogel improved dyeing uniformity, as in Experiment 1.13 compared to Experiment 1.10. The dye penetration was increased and dyeing uniformity improved by removing the Stratum Corneum, as in Experiments 1.6 and 1.10. Cleaning the skin with acetone or ethanol had only slight impact on the dye penetration depth and dyeing uniformity as indicated by tape stripping, as in Experiment 1.11. Red and yellow dyes penetrated the skin more easily than blue dye, which may be attributable to the lower molecular weight of the red and yellow dyes compared to the blue dye. Thus, embodiments of the present invention may be used for body decoration.

In the second part of the in-vitro skin penetration study, the color application included the use of a hydrogel patch and dye FD&C Red no. 40 and additional dyes, Acid Blue 1 (Blue V) and Indocyanine green, on the swine skin. All chemicals used were analytical grade, obtained from standard supply houses (Sigma, VWR, etc.). These additional dyes were chosen because they are nontoxic (approved for human injection), soluble in water, and negatively charged to be used with the cathode electrode. In these experiments, the depth of penetration was tested for the additional dyes using iontophoresis and/or electroosmosis. Additionally, extensive work was done in order to establish a shaped tattoo that is sharp and has strong color imprint. The experiments were also directed to the effect of the application duration on the tattoo life span. The following properties of the tattoo were measured to determine the effects of iontophoresis on the dyes and pigments' skin penetration: uniformity of the dyeing by observation; penetration depth by tape stripping; and water durability by washing the skin with tap water.

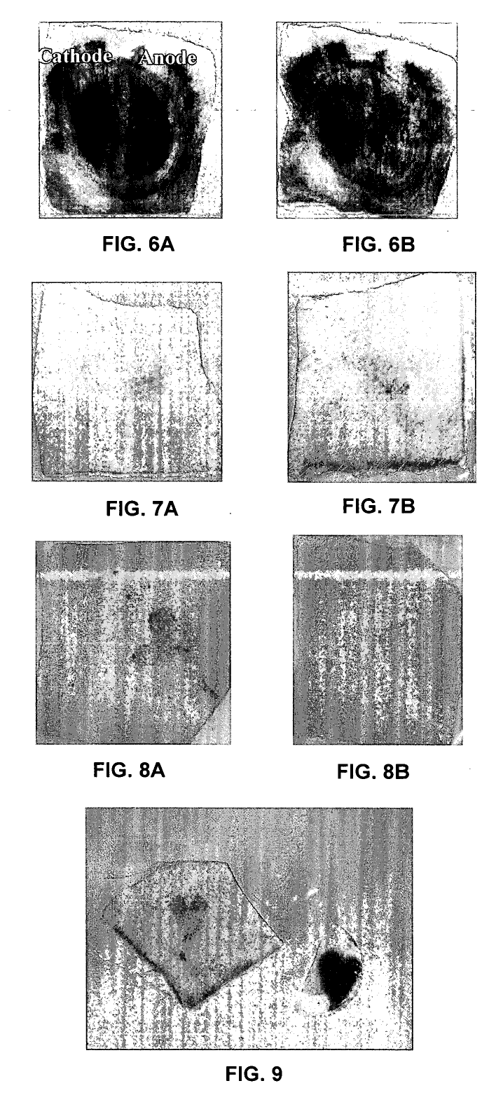

The experiments performed in this second part of the study are described below. Experiments 2.1-2.8 and 2.10-2.14 were conducted on in-vitro swine skin. Experiments 2.9, 2.15, and 2.16 were conducted on in-vivo human skin. FIGS. 6-14 illustrate the body decoration results from the experiments. Table 3 summarizes the experimental results.

Experiment 2.1

The skin penetration of 20 mg/ml Acid Blue 1 (Blue V) in ethanol with 20% double distilled water (ddw) was assayed using the vertical diffusion cells. 200 μl of the dye was added to the donor chambers and was allowed to dry on the skin. One to two hours later, 1 ml of gel containing 39.2 g ddw and 0.4 g Hydroxyethyl cellulose (Natrosol) was added to the donor chambers with (active assay) and without electric current (passive assay). Hydroxyethyl cellulose may comprise hydroxyethyl cellulose (Natrosol 250 HHBR, Hercules) at 1% wt. and water at 99% wt. In the active experiments, Ag/AgCl wires were inserted into the chamber cells and the cathode/anode were attached to them. The required current was 500 μA/cm², thus the instrument was accordingly tuned to 335 μA. Prior to the assay, the skin was cleaned with 10% lactic acid of pH≅3.5. The lactic acid solution may comprise 90% lactic acid at 10% wt., 1M sodium hydroxide at 60% wt., and water at 30% wt. The assay lasted 30 minutes and was done in duplicates. The dye penetration depth was determined by tape stripping. FIG. 6A shows the skin penetration of Blue V before the tape stripping. FIG. 6B shows the skin penetration after tape stripping; there is still Blue V color.

Experiment 2.2

In this experiment, the skin penetration of 20 mg/ml FD&C Red no. 40 (in ddw with 30% ethanol) was assayed by using a different in vitro model. Instead of conducting the experiment with vertical diffusion cells and with gel, this experiment was conducted in petri dishes and with hydrogel patches (with Ag/AgCl electrodes). The skin was cleaned with 10% lactic acid (pH≅3.5) and then was placed on PBS-soaked gauze with dermis facing the gauze. To create colored shapes, a thin paper napkin was cut to little squares, which were soaked in Red 40 solution and were placed on the skin. Two hydrogel patches, anode and cathode, were then placed on top of the red squares. In the active experiments, the patch wires were connected to a power supply tuned to 0.5 mAmp (0.5 mAmp/cm²). The assay lasted 30 minutes and was done in duplicates. The dye penetration depth was determined by tape stripping.

Experiment 2.3

In this experiment, blue heart-shaped tattoos were created. Tissue paper was cut to heart-shaped pieces that were soaked in Blue V solution (20 mg/ml in ethanol with 20% ddw). The blue heart shapes were placed on the patches' hydrogel and removed a few seconds later, leaving a blue heart-shaped imprint on the hydrogel. The assay was done in petri dishes as described in Experiment 2.2. In this assay, the skin was not cleaned with 10% lactic acid. The hydrogel patches with the blue heart-shaped imprint were placed on the skin. In the active experiments, the patch wires were connected to a power supply tuned to 0.5 mAmp (0.5 mAmp/cm²). The assay lasted 30 minutes and was done in duplicates. The dye penetration depth was determined by tape stripping.

FIG. 7A shows the blue heart-shaped tattoo created with application of electric current to non-pretreated skin. FIG. 7B shows the blue heart-shaped tattoo created without applying electric current to non-pretreated skin. The tattoo applied without electric current in FIG. 7B is fainter than that applied with electric current in FIG. 7A. In both instances, the tattoos are visible, but faint. FIG. 9 shows the blue heart-shaped imprint on the hydrogel and on the skin after application of the imprinted hydrogel.

Experiment 2.4

In this experiment, blue heart-shaped tattoos were created under the same conditions as in Experiment 2.3, except the skin was cleaned with 10% lactic acid prior to the assay. FIG. 8A shows the blue heart-shaped tattoo created with application of electric current to pretreated skin. FIG. 8B shows the blue heart-shaped tattoo created without applying electric current to pretreated skin. The tattoos applied to the pretreated skin are more visible than those applied to non-pretreated skin, as in Experiment 2.3.

Experiment 2.5

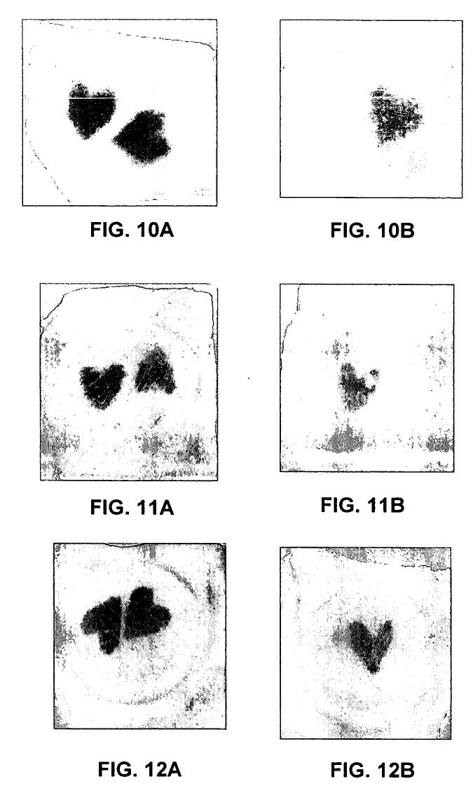

To improve the appearance of the heart-shaped tattoo, this experiment was done with a high concentration of Blue V (100 mg/ml in ddw with 20% ethanol). Other parameters were the same as described in Experiment 2.4. FIG. 10A shows the blue heart-shaped tattoos created with the high concentration ink on pretreated skin and with application of electric current. FIG. 10B shows the blue heart-shaped tattoos created with the high concentration ink on pretreated skin without application of electric current. The high concentration improves the visibility of the tattoos over those of Experiment 2.4 at the lower ink concentration.

Experiment 2.6

This experiment was conducted to determine the dye penetration depth of stretched skin, as would be the case on the subject's body area. In the vertical diffusion cells without the cell tops, the skin was stretched with the cell clamps. To increase the water content of the hydrogel in the patches, the hydrogel was soaked with ddw for 2 minutes. Excess water was then absorbed. Tissue paper heart-shaped pieces were soaked in Blue V solution (100 mg/ml in ddw). The blue heart shapes were placed on the patches' hydrogel and removed a few seconds later, leaving a blue heart-shaped imprint on the hydrogel. The hydrogel patches with the blue heart-shaped imprint were placed on the skin after the skin was cleaned with 10% lactic acid. In the active experiments, the patch wires were connected to a power supply tuned to 0.5 mAmp (0.5 mAmp/cm²). The assay lasted 30 minutes and was done in duplicates. The dye penetration depth was determined by tape stripping.

FIGS. 11A and 11B show the blue heart-shaped tattoos created with high concentration ink on stretched skin to simulate the skin on the subject's body area. FIG. 11A shows the blue heart-shaped tattoos created with the highconcentration ink on pretreated stretched skin and with application of electric current. FIG. 11B shows the blue heart-shaped tattoos created with the high concentration ink on pretreated skin without application of electric current. The stretched skin provided acceptable ink penetration depth and improved tattoo visibility, as compared to the tattoos in Experiment 2.5.

Experiment 2.7

The skin penetration of 2 mg/ml Indocyanine green in ethanol with 40% ddw was assayed using the vertical diffusion cells. 200 μl of the dye was added to the donor chambers and was allowed to dry on the skin. One or two hours later, 1 ml of gel containing 39.2 g ddw and 0.4 g Hydroxyethyl cellulose (Natrosol) was added to the donor chambers with (active assay) and without (passive assay) electric current. In the active experiments, Ag/AgCl wires were inserted into the chamber cells and the cathode/anode were attached to them. The required current was 500 μA/cm2, thus the power supply was accordingly tuned to 320 μA (for 0.64 cm²). Prior to the assay, the skin was cleaned with 10% lactic acid of pH≅3.5. The assay lasted 30 minutes and was done in duplicates. The dye penetration depth was determined by tape stripping.

Experiment 2.8

Blue heart-shaped tattoos were created, as described before in Experiment 6. This experiment was conducted to sharpen the tattoo by soaking the hydrogel in ddw for only few seconds. FIG. 12A shows the blue heart-shaped tattoos created with the ddw-soaked hydrogel and application of electric current. FIG. 12B shows the blue heart-shaped tattoos created with the ddw-soaked hydrogel and without application of electric current. These tattoos were sharper than those of Experiment 2.6 that did not use ddw-soaked hydrogel.

Experiment 2.9

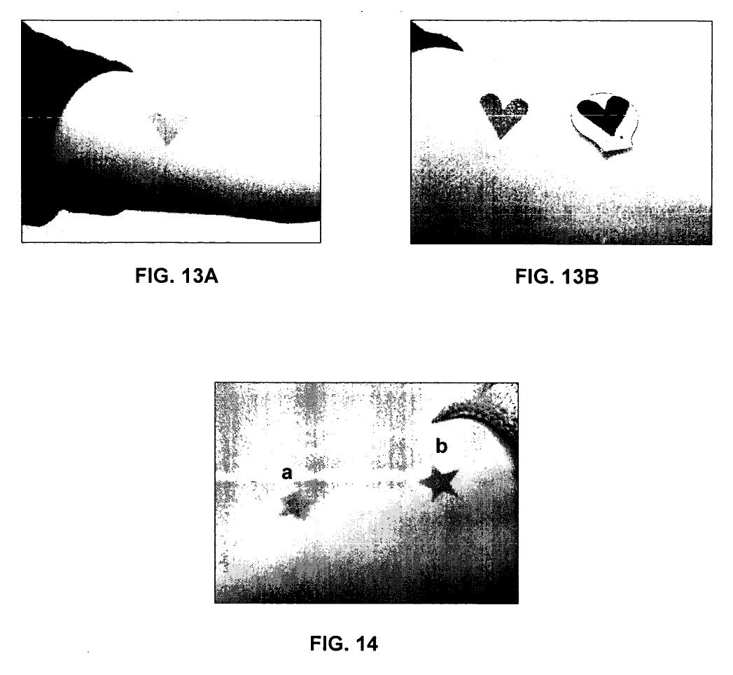

This experiment was the first of three conducted on in vivo human skin. Tissue paper was cut to heart-shaped pieces that were soaked in Blue V solution (50 mg/ml in ddw). The blue heart shapes were placed on the patches' hydrogel and removed a few seconds later, leaving a blue heart-shaped imprint on the hydrogel. The left forearm of the volunteer was cleaned with 10% lactic acid of pH≅3.5. Two separate patches (one with the heart-shaped imprint) with wires were placed on the forearm. The wires were connected to a power supply in the way that the imprinted heart-shaped form was placed under the cathode electrode. The power supply was set to 3V. The assay lasted 20 minutes. FIG. 13A shows the blue heart-shaped tattoo applied by the imprinted hydrogel. FIG. 13B shows the tattoo and the imprinted hydrogel. The tattoo has a uniform, strong color. The tattoo completely faded after three days. See Table 3.

Experiment 2.10

To improve the skin penetration of Indocyanine green, this experiment was conducted with a higher concentration (25 mg/ml in ethanol) of the dye than was used in Experiment 2.7. Other parameters were the same as described in Experiment 2.7. The results in Table 3 indicate that the penetration depth of the green dye was relatively unaffected by the increased dye concentration.

Experiment 2.11

To increase the blue heart-shaped penetration depth, 200 mg/ml Blue V (in ddw with 20% ethanol) was used. The assay lasted 20 minutes to avoid decrease in the current due to increase in the voltage. Other parameters were the same as described in Experiment 6. Instead of using tape stripping to determine the dye penetration depth, the skin was washed with tap water to determine the dye's water durability. Washing the skin removed almost all the dye.

Experiment 2.12

In this experiment, a red heart-shaped tattoo was created. Tissue paper was cut to heart-shaped pieces that were soaked in FD&C Red no. 40 solution (100 mg/ml in ddw). The red heart shapes were placed on the patches' hydrogel and removed a few seconds later, leaving a red heart-shaped imprint on the hydrogel. The hydrogel patches with the red heart-shaped imprint were placed on the skin, after the skin was cleaned with 10% lactic acid, and then placed in the vertical diffusion cells without the cell tops. In the active experiments, the patch wires were connected to a power supply tuned to 0.5 mAmp (0.5 mAmp/cm²). The assay lasted 20 minutes and was done in duplicates. To determine the dye's water durability, the skin was washed with tap water. Washing the skin removed almost all the dye.

Experiment 2.13

In this experiment, the patches' hydrogel content was changed. Instead of KCl, the hydrogel contained NaCl. 100 mg/ml Blue V in ddw with 10% ethanol was used to create the blue heart-shaped imprint. The assay lasted 20 minutes and was done in duplicates. Other parameters were the same as described in Experiment 2.6. The penetration depth was lower with NaCl than KCl in Experiment 2.6.

Experiment 2.14

To concentrate the current flow through the heart-shaped imprint area, a polyester decorative template was interposed between the hydrogel and the skin. Siliconized polyester (25 micron) was cut in the middle, forming heart-shaped template. The polyester template was placed on the hydrogel and then Blue V (100 mg/ml in ddw with 10% ethanol) was added, creating a blue heart-shaped imprint on the hydrogel. Excess color was removed using tissue paper. The skin was cleaned with 10% lactic acid. Then the hydrogel with the blue heart-shaped imprint and with the polyester template disposed thereon was placed on the skin. In the active experiments, the patch wires were connected to a power supply tuned to 0.5 mAmp (0.5 mAmp/cm²). The assay lasted 40 minutes and was done in duplicates. The skin was washed with tap water to determine the dye's water durability. The durability was high.

Experiment 2.15

This experiment was the second of three conducted on human skin. In this experiment, a blue star-shaped imprint was created on the hydrogel by using a siliconized polyester star-shaped template. 50 μl of Blue V (50 mg/ml in ddw) was added to the hydrogel in the star cut area and excess dye removed, leaving a blue star-shaped imprint on the hydrogel. The left forearm of the volunteer was cleaned with 10% lactic acid at pH≅3.5. Two separate patches (with the star-shaped imprint and siliconized polyester) with wires were placed on the forearm. The wires were connected to a power supply in the way that the imprinted star form with the polyester was placed under the cathode electrode. The power supply was tuned to 0.04 mAmp. The assay lasted 40 minutes. The dye completely faded after 10 days.

Experiment 2.16

This experiment was the third of three conducted on human skin. This experiment was conducted to study the tattoo life span and the effect of the decorative template thereto. To prolong the tattoo life span, the application time of the dye to the skin by iontophoresis was extended to 60 minutes. To study the effect of the template on the tattoo life span, two double hydrogel patches (with cathode and anode) were placed on the volunteer's forearm. One of the patches was with the polyester template and the other one without. Other parameters were the same as described in Experiment 2.15. FIG. 14 shows tattoo “a” applied with an imprinted hydrogel with the polyester template and tattoo “b” applied with the imprinted hydrogel without the template. With the template, the dye faded after 10 days. Without the template, the dye faded after 7 days. See Table 3.

Table 3 shows the results of Experiments 2.1-2.16. According to the results, iontophoresis increased the skin penetration depth of Blue V under the cathode electrode, as indicated in Experiment 2.1 and FIG. 6. Using embodiments of the present invention, tattoos were successfully created on a body area by imprinting the decorative form on the hydrogel, using either tissue papers or polyesters as templates. Cleaning the skin with 10% lactic acid (pH≅3.5) strengthened the skin dyeing, as indicated in Experiment 2.4 compared to Experiment 2.3 and FIG. 8 compared to FIG. 7. Increasing the dye concentration contributed to increased penetration depth for some dyes, as indicated in Experiment 2.5 compared to Experiment 2.4 and FIG. 10 compared to FIG. 8. The heart shape imprint was less sharp in the petri dish model (as indicated in Experiment 2.6 compared with Experiment 2.5, which apparently was because the skins in this model were not stretched. The penetration depth of indocyanine green was not affected by iontophoresis, as indicated by Experiments 2.7 and 2.10. Adding water to the hydrogel had a negative effect on the tattoo imprint sharpness, as indicated in Experiment 2.6 compared with Experiment 2.8. Longer iontophoretic application prolonged the tattoo life span, as indicated in Experiment 2.15 compared to Experiment 2.9. Adding the polyester template that concentrated the current flow through the imprint area prolonged the tattoo life span, as indicated in Experiment 16. Finally, the hydrogel with the polyester on it was less adhesive, and thus the tattoo formed using this arrangement was slightly blurred. (See FIG. 14.) Thus, embodiments of the present invention may be used for body decoration.

The colors that were found to be most suitable for application to a body area of a subject according to the present invention were FD&C Red No. 40, FD&C Yellow No. 6 and Acid Blue 1 (Blue V). All of these colors are negatively charged. Accordingly, it is preferable that the cathode electrode (which is negatively charged, and thus drives negatively charged color formulations into a body area of a subject) be used to promote the penetration of a color formulation into a body area of a subject.

The embodiments of the present invention are not limited to applying body decoration. These embodiments may also be applied to remove body decoration. In such an application, reverse iontophoresis may be applied to draw the tattoo ink from the body area. A patch may be placed on the tattoo area and electric current applied to drive the ink from the body area toward the appropriate electrode and into a layer that would normally hold the color formulation. Alternatively, normal iontophoresis may be used to apply a substance to dissolve, discolor, or otherwise remove the tattoo ink. Optionally, the substance may be applied through a tattoo-shaped template to the tattoo area and the electric current applied through the template until the tattoo is no longer visible.

In an alternate application, embodiments of the present invention may be used to tattoo animals. These tattoos may be used for stock control, as an alternative to painful branding. These tattoos may also be used as a fashion feature on pets, for example. In such an application, color formulation may be applied to the animal's body through the desired template, e.g., a template in the shape of a rancher's brand. The patch may be applied to promote penetration of the color formulation into the animal's body.

In another application, embodiments of the present invention may be used to apply semi-permanent cosmetics. In this application, color formations may be applied to a subject's face, as rogue, eye make-up, lipstick, lip liner, eye brow liner etc. The subject may apply a decorative template outlining the facial area to be colored and then apply by hand or with a cosmetic delivery device the desired cosmetic. The subject may then apply the patch to promote penetration of the color formulation into the facial area. The subject may control the penetration depth and, hence, the durability of the color formulation by adjusting the electric current supplied by the patch, the duration of the application, and the colors chosen. Alternatively, the subject may apply the patch with the decorative template integrated therewith in a seemingly single-step application. This application advantageously allows the subject to wear semi-permanent cosmetics without having to reapply cosmetics daily. Similarly, color formulations may be applied to a subject's fingernails and toenails.

Alternatively, skin treatments may be applied using embodiments of the present invention. For example, therapeutic treatments, such as scar healing, wrinkle reduction, antibiotic treatment, etc., may be applied to the body of a subject through a template and/or patch as previously described. The conductive composition such as hydrogel can optionally contain the color/dye and an active medicinal ingredient. Alternatively, the patch can optionally contain a chamber containing the color dye and active medicinal ingredient without a conductive composition. Such a therapeutic tattoo is configured to simultaneously provide a decorative tattoo and therapeutic treatment. For example, a therapeutic tattoo patch could be used on a spot, wherein the tattoo will facilitate concealing the spot and the active ingredient will treat the spot. Other non-limiting examples of use of such a patch include treatment of acne, wrinkles, skin discoloration, excessive skin coloration, skin puffiness, scarring, dry skin, oily skin, imbalance of skin pH, and a combination thereof.

The above is a detailed discussion of the preferred embodiments of the invention. The full scope of the invention to which applicants are entitled is defined by the claims hereinafter. It is intended that the scope of the claims may cover other embodiments than those described above and their equivalents.

TABLE 2

| | | | | | | Voltage | Current | Depth of penetration |
|---|---|---|---|---|---|---|---|---|
| Experiment | Remarks | Dye | Concentration | Assay | Time | (Volts) | (mAmp) | (Tape strips = TS) |
| 1.1 | The current was limited | Blue | 10 mg/ml in ddw | Active | t0 | ˜3.05 | ˜0.335 | None |
| | to 335 mA (500 μA/cm²). | no. 1 | | | t30 | ˜4.9 | ˜0.335 | |
| | Full skin | | | Passive | | None | None | None |
| | | Yellow | 20 mg/ml in ddw | Active | t0 | ˜3.00 | ˜0.335 | None |
| | | no. 6 | | | t30 | ˜4.2 | ˜0.335 | |
| | | | | Passive | | None | None | None |
| | | Red | 20 mg/ml in ddw | Active | t0 | ˜3.00 | ˜0.334 | None |
| | | no. 40 | | | t30 | ˜4.5 | ˜0.335 | |
| | | | | Passive | | None | None | None |
| 1.2 | The current was unlimited. | Blue | 100 mg/ml in ddw | Active | t0 | ˜7.9 | ˜1.3 | None |
| | Full skin | no. 1 | | | t30 | ˜9.4 | ˜1.0 | |
| | | | | Passive | | None | None | None |
| | | Yellow | 60 mg/ml in ddw | Active | t0 | ˜7.9 | ˜0.860 | None |
| | | no. 6 | (Was slightly insoluble) | | t30 | ˜8.6 | ˜0.195 | |
| | | | | Passive | | None | None | None |
| | | Red | 60 mg/ml in ddw | Active | t0 | ˜8.5 | ˜0.615 | None |
| | | no. 40 | | | t30 | ˜8.6 | ˜0.443 | |
| | | | | Passive | | None | None | None |
| 1.3 | The current was unlimited. | Blue | 100 mg/ml in ddw with 30% | Active | t0 | ˜8.6 | ˜1.2 | None |
| | Ethanol was added to the | no. 1 | ethanol | | t30 | ˜8.5 | ˜0.436 | |
| | color solution. | | | Passive | | None | None | None |
| | Full skin | Yellow | 25 mg/ml in ddw with 30% | Active | t0 | ˜3.5 | ˜1.3 | None |
| | | no. 6 | ethanol | | t30 | ˜9.4 | ˜0.440 | |
| | | | (Poor solubility) | Passive | | None | None | None |
| | | Red | 100 mg/ml in ddw with 30% | Active | t0 | ˜7.0 | ˜1.3 | None |
| | | no. 40 | ethanol | | t30 | ˜8.0 | ˜0.430 | |
| | | | | Passive | | None | None | None |
| 1.4 | The current was unlimited. | Blue | 40 mg/ml in ethanol + 20% | Active | t0 | ˜6.0 | ˜1.2 | Spot dyeing |
| | The assay was done with | no. 1 | ddw | | t30 | ˜7.0 | ˜1.0 | Cathode - TS# 8 |
| | dried color + gel. | | | | | | | Anode - TS# 6 |
| | Full skin | | | Passive | | None | None | TS# 6 |
| 1.5 | The current was unlimited. | Blue | In 100% ethanol | Active | t0 | ˜8.2 | ˜0.63 | Spot dyeing |
| | The assay was done with | color | | | t30 | ˜8.4 | ˜0.350 | Cathode - TS# 10 |
| | dried color + gel. | mixture | | | | | | Anode - TS# 5 |
| | Full skin | | | Passive | | None | None | TS# 5 |
| 1.6 | The current was unlimited. | Blue | 40 mg/ml in ethanol + 20% | Active | t0 | ˜4.5 | ˜1.2 | Uniform dyeing |
| | The assay was done with | no. 1 | ddw | | t30 | ˜8.4 | ˜0.4 | Cathode - TS# >15 |
| | dried color + gel. | | | | | | | Anode - TS# 10 |
| | The skin was without | | | Passive | | None | None | TS# 11 |
| | Stratum Corneum. | | | | | | | |
| 1.7 | The current was unlimited. | Blue | 40 mg/ml in ethanol + 20% | Active | t0 | ˜8.4 | ˜0.85 | Spot dyeing |
| | The assay was done with | no. 1 | ddw | | t30 | ˜9.0 | ˜0.3 | Cathode - TS# 7 |
| | dried color + gel. | | | | | | | Anode - TS# 4 |
| | The skin was cleaned | | | Passive | | None | None | TS# 4 |
| | with Acetone. | | | | | | | |
| | The skin was without | | | | | | | |
| | Stratum Corneum. | | | | | | | |
| 1.8 | The current was unlimited. | Yellow | 20 mg/ml in ethanol + 30% | Active | t0 | ˜5.5 | ˜1.3 | Spot dyeing |
| | The assay was done with | no. 6 | ddw | | t30 | ˜8.4 | ˜0.8 | Cathode - TS# >15 |
| | dried color + gel. | | (Solubility was achieved by | | | | | Anode - TS# 4 |
| | The skin was cleaned with | | heating the solution) | Passive | | None | None | TS# 4 |
| | 70% ethanol. | Red | 20 mg/ml in ethanol + 30% | Active | t0 | ˜8.5 | ˜0.615 | Spot dyeing |
| | Full skin | no. 40 | ddw | | t30 | ˜8.6 | ˜0.443 | Cathode - TS# >15 |
| | | | (Solubility was achieved by | | | | | Anode - TS# 5 |
| | | | heating the solution) | Passive | | None | None | TS# 5 |
| 1.9 | The current was limited to | Yellow | 20 mg/ml in ethanol + 30% | Active | t0 | ˜2.7 | ˜0.335 | Spot dyeing |
| | 335 mA (500 μA/cm²). The | no. 6 | ddw (Solubility was achieved | | t30 | ˜2.4 | ˜0.335 | Cathode - TS# 4 |
| | assay was done with dried | | by heating the solution) | | | | | Anode - TS# 4 |
| | color + gel. The skin was | | | Passive | | None | None | TS# 4 |
| | cleaned with 70% ethanol. | | | | | | | |
| | Full skin | | | | | | | |
| 1.10 | The current was limited to | Blue | 40 mg/ml in ethanol + 20% | Active | t0 | ˜2.4 | ˜0.335 | Spot dyeing |
| | 335 mA (500 μA/cm²). The | no. 1 | ddw | | t30 | ˜2.2 | ˜0.335 | Cathode - TS# >7 |
| | assay was done with dried | | | | | | | Anode - TS# 7 |
| | color + gel. | | | Passive | | None | None | TS# 7 |
| | The skin was without | Yellow | 20 mg/ml in ethanol + 30% | Active | t0 | ˜1.6 | ˜0.335 | Spot dyeing but after 1 |
| | Stratum Corneum. | no. 6 | ddw | | t30 | ˜1.6 | ˜0.335 | hour uniform dyeing was |
| | | | (Solubility was achieved by | | | | | observed |
| | | | heating the solution) | | | | | Cathode - TS# >10 |
| | | | | | | | | Anode - TS# 6 |
| | | | | Passive | | None | None | TS# 6 |
| | | Red | 20 mg/ml in ethanol + 30% | Active | t0 | ˜2.1 | ˜0.335 | Spot dyeing |
| | | no. 40 | ddw | | t30 | ˜2.0 | ˜0.335 | Cathode - TS# >9 |
| | | | (Solubility was achieved by | | | | | Anode - TS# 5 |
| | | | heating the solution) | Passive | | None | None | TS# 5 |
| 1.11 | The current was limited to | Red | 20 mg/ml in ethanol + 30% | Active | t0 | ˜3.0 | ˜0.335 | Spot dyeing |
| | 335 mA (500 μA/cm²). The | no. 40 | ddw (Solubility was | | t30 | ˜3.4 | ˜0.335 | Cathode - TS# >7 |
| | assay was done with | | achieved by heating the | | | | | Anode - TS# 4 |
| | dried color + gel. The skin | | solution) | Passive | | None | None | TS# 4 |
| | was cleaned with | | | | | | | |
| | 10% lactic acid. | | | | | | | |
| | Full skin | | | | | | | |
| 1.12 | The current was limited to | Red | 20 mg/ml in ethanol + 30% | Active | t0 | ˜3.2 | ˜0.335 | Spot dyeing |
| | 335 mA (500 μA/cm²). The | no. 40 | ddw (Solubility was | | t30 | ˜2.7 | ˜0.335 | Cathode - TS# >7 |
| | assay was done with dried | | achieved by heating the | | | | | Anode - TS# 5 |
| | color + gel. The skin was | | solution) | Passive | | None | None | TS# 5 |
| | cleaned with 5% lactic acid. | | | | | | | |
| | Full skin | | | | | | | |
| 1.13 | The current was limited | Red | 20 mg/ml in ethanol + 30% | Active | t0 | ˜0.65 | ˜0.335 | Spot dyeing |
| | to 335 mA. After 7 min the | no. 40 | ddw (Solubility was | | t30 | ˜2.9 | ˜0.08 | Cathode - TS# >7 |
| | voltage was limited to 3 V. | | achieved by heating the | | | | | Anode - TS# 4 |
| | The assay was done with | | solution) | Passive | | None | None | TS# 4 |
| | dried color + hydrogel. | | | | | | | |
| | The skin was without | | | | | | | |
| | Stratum Corneum. | | | | | | | |

TABLE 3

| | | | | | | Voltage | Current | Depth of penetration |
|---|---|---|---|---|---|---|---|---|
| Experiment | Description | Dye | Concentration | Assay | Time | (Volts) | (mAmp) | (Tape strips = TS) |
| 2.1 | The current = 500 μA/cm² | Blue V | 20 mg/ml in | Active | t0 | ˜2.8 | 0.335 | Uniform dyeing |
| | The skin was cleaned with | | ethanol with | | t30 | ˜2.4 | 0.335 | Cathode - TS# >10 |
| | 10% lactic acid | | 20% ddw | | | | | Anode - TS# 9 |
| | | | | Passive | | None | None | TS# 8 |
| 2.2 | The current = 500 μA/cm² | Red | 20 mg/ml in | Active | t0 | ˜3.8 | 0.5 | Uniform weak dyeing |
| | The skin was cleaned with | no. 40 | ddw with | | t30 | ˜8.4 | ˜0.195 | Cathode - TS# >7 |
| | 10% lactic acid | | 30% ethanol | | | | | Anode - TS# 6 |
| | Petri dish model | | | Passive | | None | None | None |
| 2.3 | Blue heart shape | Blue V | 20 mg/ml in | Active | t0 | ˜1.0 | 0.5 | Uniform heart shape |
| | The current = 500 μA/cm² | | ethanol with | | t30 | ˜2.2 | 0.5 | dyeing. |
| | Petri dish model | | 20% ddw | | | | | Very faint dye. |
| | | | | | | | | Cathode - TS# >6 |
| | | | | | | | | Anode - TS# 5 |
| | | | | Passive | | None | None | TS# 3 |
| 2.4 | Heart shape | Blue V | 20 mg/ml in | Active | t0 | ˜4.1 | 0.5 | Uniform heart shape |
| | The current = 500 μA/cm² | | ethanol with | | t30 | ˜5.1 | 0.5 | dyeing. |
| | The skin was cleaned with | | 20% ddw | | | | | Faint color. |
| | 10% lactic acid | | | | | | | Cathode - TS# >7 |
| | Petri dish model | | | | | | | Anode - TS# 4 |
| | | | | Passive | | None | None | TS# 4 |
| 2.5 | Heart shape | Blue V | 100 mg/ml in | Active | t0 | ˜2.8 | 0.5 | Uniform heart shape |
| | The current = 500 μA/cm² | | ethanol with | | t30 | ˜8.1 | 0.5 | dyeing. |
| | The skin was cleaned with | | 20% ddw | | | | | Strong color. |
| | 10% lactic acid | | | | | | | Cathode - TS# >6 |
| | Petri dish model | | | | | | | Anode - TS# 5 |
| | | | | Passive | | None | None | TS# 4 |
| 2.6 | Heart shape | Blue V | 100 mg/ml in | Active | t0 | ˜2.8 | 0.5 | Uniform sharp heart shape |
| | The current = 500 μA/cm² | | ddw | | t30 | ˜8.1 | 0.5 | dyeing. |
| | The skin was cleaned with | | | | | | | Strong color. |
| | 10% lactic acid | | | | | | | Cathode - TS# >6 |
| | | | | | | | | Anode - TS# 5 |
| | | | | Passive | | None | None | TS# 4 |
| 2.7 | The current = 500 μA/cm² | Green | 2 mg/ml in | Active | t0 | ˜8.5 | ˜0.25 | Uniform dyeing |
| | The skin was cleaned with | | ethanol with | | t30 | ˜8.5 | ˜0.30 | Cathode - TS# 2 |
| | 10% lactic acid | | 40% ddw | | | | | Anode - TS# 2 |
| | | | | Passive | | None | None | TS# 2 |
| 2.8 | Heart shape | Blue V | 100 mg/ml in | Active | t0 | ˜2.0 | 0.5 | Uniform heart shape |
| | The current = 500 μA/cm² | | ddw | | t20 | ˜3.4 | 0.5 | dyeing. |
| | The skin was cleaned with | | | | | | | Faint dye. |
| | 10% lactic acid | | | | | | | Cathode - TS# >7 |
| | | | | | | | | Anode - TS# 4 |
| | | | | Passive | t25 | ˜8.4 | 0.18 | TS# 4 |
| 2.9 | Heart shape | Blue V | 50 mg/ml in | Active | t0 | 3.0 | 0.013 | Uniform heart shape |
| | The voltage = 3 volt | | ddw | | t20 | 3.0 | 0.014 | dyeing. |
| | The skin was cleaned with | | | | | | | Strong color. |
| | 10% lactic acid | | | | | | | The dye completely faded |
| | Human study | | | | | | | after 3 days. |
| 2.10 | The current = 500 μA/cm² | Green | 25 mg/ml in | Active | t0 | ˜8.0 | ˜0.270 | Uniform dyeing |
| | The skin was cleaned with | | 100% ethanol | | t30 | ˜6.5 | ˜0.320 | Cathode - TS# 3 |
| | 10% lactic acid | | | | | | | Anode - TS# 3 |
| | | | | Passive | | None | None | TS# 2 |
| 2.11 | Heart shape | Blue V | 200 mg/ml in | Active | t0 | ˜5.6 | 0.499 | Uniform heart shape |
| | The current = 500 μA/cm² | | ddw with 20% | | t20 | ˜7.52 | 0.499 | dyeing. |
| | The skin was cleaned with | | ethanol | | | | | Strong color. |
| | 10% lactic acid | | | | | | | Washing the skin |
| | | | | | | | | removed almost all the |
| | | | | | | | | dye. |
| | | | | Passive | | None | None | Uniform heart shape |
| | | | | | | | | dyeing. |
| | | | | | | | | Less color compared to |
| | | | | | | | | the active. |
| | | | | | | | | Washing the skin |
| | | | | | | | | removed all the dye. |
| 2.12 | Heart shape | Red | 100 mg/ml in | Active | t0 | ˜3.1 | 0.499 | Uniform heart shape |
| | The current = 500 μA/cm² | no. 40 | ddw | | t20 | ˜3.4 | 0.499 | dyeing. |
| | The skin was cleaned with | | | | | | | Strong color. |
| | 10% lactic acid | | | | | | | Washing the skin |
| | | | | | | | | removed almost all the |
| | | | | | | | | dye. |
| | | | | Passive | | None | None | Uniform heart shape |
| | | | | | | | | dyeing. |
| | | | | | | | | Less color compared to |
| | | | | | | | | the active. |
| | | | | | | | | Washing the skin |
| | | | | | | | | removed all the dye. |
| 2.13 | Heart shape | Blue V | 100 mg/ml in | Active | t0 | ˜1.9 | ˜0.50 | Uniform dyeing |
| | Hydrogel with NaCl | | ddw with 10% | | t18 | ˜1.9 | ˜0.50 | Cathode - TS# >4 |
| | The current = 500 μA/cm² | | ethanol | | t20 | ˜8.4 | ˜0.22 | Anode - TS# 3 |
| | The skin was cleaned with | | | Passive | | None | None | TS# 3 |
| | 10% lactic acid | | | | | | | |
| 2.14 | Star shape | Blue V | 100 mg/ml in | Active | t0 | ˜5.3 | 0.5 | Uniform heart shape |
| | With polyester as a | | ddw with 10% | | t30 | ˜5.13 | 0.5 | dyeing. |
| | partition | | ethanol | | t40 | ˜16.3 | 0.15 | Strong color. |
| | The current = 500 μA/cm² | | | | | | | Washing the skin didn't |
| | The skin was cleaned with | | | | | | | remove all the dye. |
| | 10% lactic acid | | | Passive | | None | None | Uniform heart shape |
| | | | | | | | | dyeing. |
| | | | | | | | | Less color compared to |
| | | | | | | | | the active. |
| | | | | | | | | Washing the skin |
| | | | | | | | | removed all the dye. |
| 2.15 | Star shape | Blue V | 50 mg/ml in | Active | t0 | ˜7.1 | 0.04 | Uniform star shape dyeing. |
| | With polyester as a | | ddw | | | | | Strong color. |
| | partition | | | | | | | The dye completely faded |
| | The current = 500 μA/cm² | | | | | | | after 10 days. |
| | The skin was cleaned with | | | | t40 | ˜6.5 | 0.04 | |
| | 10% lactic acid | | | | | | | |
| | Human study | | | | | | | |
| 2.16 | Star shape | Blue V | 50 mg/ml in | Active | t0 | ˜9.4 | 0.04 | Uniform star shape dyeing. |
| | With and without | | ddw | With | t60 | ˜6.7 | 0.04 | Strong color. |
| | polyester as a partition | | | polyester | | | | The dye completely faded |
| | The current = 500 μA/cm² | | | | | | | after 10 days. |
| | The skin was cleaned with | | | Active | t0 | ˜9.4 | 0.04 | Uniform star shape dyeing. |
| | 10% lactic acid | | | Without | t60 | ˜5.2 | 0.04 | Strong color. |
| | Human study | | | polyester | | | | The dye completely faded |
| | | | | | | | | after 7 days. |

What is claimed is:

1. A kit for applying a body decoration to a subject, comprising:
a color formulation;
means for applying said color formulation to a body area of a subject; and
an electrically powered patch that promotes penetration of said color formulation into said body area.

2. The kit of claim 1, wherein said patch is an iontophoretic patch.

3. The kit of claim 2, wherein said patch comprises:
a first electrode;
a second electrode separated from said first electrode; and
a power supply supported on a base member.

4. The kit of claim 3, wherein said power supply comprises at least one electrochemical cell.

5. The kit of claim 3 wherein said power supply further comprises at least one electronic component.

6. The kit of claim 4, wherein said electrochemical cell is thin and flexible.

7. The kit of claim 6, wherein said electrochemical cell is up to 4 mm thick.

8. The kit of claim 6, wherein said electrochemical cell is up to 2 mm thick.

9. The kit of claim 6, wherein said electrochemical cell is up to 1 mm thick.

10. The kit of claim 3, wherein said first electrode is an anode and said second electrode is a cathode.

11. The kit of claim 4, wherein said electrochemical cell comprises:
a first layer of insoluble negative pole;
a second layer of insoluble positive pole; and
a third layer of aqueous electrolyte disposed between said first and second layers and including (a) a deliquescent material for keeping said electrochemical cell wet at all times; (b) an electroactive soluble material for obtaining ionic conductivity; and (c) a water-soluble polymer for obtaining a desired viscosity for adhering said first and second layers to said third layer.

12. The kit of claim 4, wherein said electrochemical cell has open architecture.

13. The kit of claim 10, wherein said patch comprises an active electrode, and said active electrode is said anode, said cathode or both said anode and said cathode.

14. The kit of claim 2, wherein said color formulation is selected from the group consisting of a positively charged color formulation, a negatively charged color formulation, a neutral color formulation, and a combination thereof.

15. The kit of claim 1, wherein said color formulation includes at least one of the group consisting of a dye, a pigment, an ink, and a combination thereof.

16. The kit of claim 1, wherein said color formulation is selected from the group consisting of single colors, combinations of colors, mixtures of colors, fluorescent colors, glitter, metallic colors, melanin, skin colors, and any other desirable color suitable for body decoration.

17. The kit of claim 1, wherein said means for applying comprises a stencil having a decorative shape therein.

18. The kit of claim 1, wherein said means for applying comprises a transferable sheet with a decorative shape thereon.

19. The kit of claim 3, wherein said means for applying comprises said first electrode or said second electrode formed in a decorative shape.

20. The kit of claim 1, further comprising:
a conductive layer comprising a conductive composition disposed between said patch and said body area, for providing a conductive interfacing layer between said patch and said body area.

21. The kit of claim 20, wherein said conductive composition is a conductive adhesive.

22. The kit of claim 21, wherein said conductive adhesive is a hydrogel.

23. The kit of claim 20, wherein said color formulation is contained in said conductive composition.

24. The kit of claim 20, wherein said patch comprises an active electrode and a counter electrode, and said conductive layer is disposed on said active electrode.

25. The kit of claim 20, wherein said patch further comprises an additional conductive layer disposed on said counter electrode.

26. The kit of claim 24, wherein said color formulation is contained in said conductive composition, and said conductive layer is printed on said active electrode with said color formulation in the desired decorative form.

27. The kit of claim 24, wherein said conductive layer disposed on said active electrode is preprinted with said color formulation in the desired decorative form, and is not affixed to the active electrode.

28. The kit of claim 24, wherein said conductive layer disposed on said active electrode is in the shape of the body decoration.

29. The kit of claim 3, wherein said means for applying comprises a decorative template that covers one or more regions of said body area.

30. The kit of claim 29, wherein said decorative template is non-conductive.

31. The kit of claim 29, wherein said decorative template is made from polyester.

32. The kit of claim 29, wherein said decorative template comprises a cutout in the shape of the body decoration.

33. The kit of claim 29, wherein said decorative template limits application of said color formulation to those regions of said body area not covered by said template, and limits the electric current flowing from one of the electrodes to said body area to those regions of said body area not covered by said template.

34. The kit of claim 1, wherein said body decoration is a tattoo.

35. The kit of claim 1, wherein said body decoration is temporary.

36. The kit of claim 35, wherein said body decoration lasts from about 1 day to about six months.

37. The kit of claim 1, wherein said body decoration is permanent.

38. The kit of claim 1, wherein said body decoration is semi-permanent make-up.

39. The kit of claim 1, wherein said patch is thin and flexible.

40. The kit of claim 1, wherein said kit is for home use.

41. The kit of claim 1, wherein said patch is less than about 10 mm thick.

42. The kit of claim 3, wherein said power supply comprises a plurality of electrochemical cells.

43. The kit of claim 3, wherein said power supply provides a duty cycle and pulse partition rate of between about 10% and about 90%.

44. The kit of claim 3, wherein said power supply provides a frequency of from about 1 Hz to about 100 Hz.

45. The kit of claim 1, wherein said patch facilitates providing an electric current selected from the group consisting of dc current, pulsed current, ac current, and a combination thereof.

46. The kit of claim 1, wherein said patch facilitates supplying an electric current through said means for applying to promote penetration of said color formulation into said body area.

47. The kit of claim 46, wherein said patch supplies electric current in the range of from about 0.02 mAmp/cm² to about 0.5 mAmp/cm².

48. The kit of claim 4 wherein said electrochemical cell supplies a voltage in a range of from about 0.5V to about 100V.

49. The kit of claim 3, wherein said power supply provides a voltage in a range of from about 0.5V to about 12V.

50. The kit of claim 49, wherein said voltage is adjusted to promote penetration of said color formulation into said body area.

51. The kit of claim 49, wherein said voltage is adjusted to minimize body area irritation.

52. The kit of claim 49, wherein said voltage is adjusted to maximize the amount of said color formulation remaining in said body area after said patch is used to apply a body decoration to a subject.

53. The kit of claim 1, wherein the penetration and durability of said body decoration is controlled by at least one parameter selected from the group consisting of iontophoresis parameters, pre-treatment parameters, post-treatment parameters, dye concentration, type of dye, the duration of application of said patch, characteristics of use of a decorative template, fixation of said patch, and a combination thereof.

54. The kit of claim 1, wherein said body area is selected from the group consisting of skin, nails, teeth, and a combination thereof.

55. The kit of claim 3, wherein said first electrode is made of a conductive material selected from the group consisting of silver, silver chloride, graphite, zinc, platinum, carbon, or a combination thereof.

56. The kit of claim 3, wherein said first electrode and said second electrode are printed electrodes.

57. The kit of claim 1, where sad patch further comprises attachment means.

58. The kit of claim 1, wherein said patch is a printed patch.

59. The kit of claim 1, wherein said subject is an animal other than a human being.

60. The kit of claim 59, wherein said body decoration is a mark used to identify animals.

61. The kit of claim 1, further comprising an active medicinal ingredient.

62. The kit of claim 1, further comprising a pre-treatment composition.

63. The kit of claim 62, where said pre-treatment composition is a peeling composition.

64. The kit of claim 63, wherein said peeling composition is lactic acid in a percentage from about 2% to about 20%.

65. The kit of claim 1, further comprising a post-treatment composition.

66. The kit of claim 65, wherein said post-treatment composition is a sealant.

67. An iontophoretic patch for applying a body decoration to a subject comprising:
(a) a substrate base layer;
(b) a first electrode attached to said substrate base layer, wherein said first electrode is an active electrode;
(c) a second electrode attached to said substrate base layer;
(d) a power supply attached to said electrodes for supplying current to said electrodes;
(e) a conductive layer disposed on said first electrode for providing a conductive interface between said first electrode and a body area of said subject; and
(f) a color formulation on said first electrode;
wherein said patch promotes penetration of said color formulation into said body area to form said body decoration.

68. The patch of claim 67, further comprising an additional conductive layer disposed on said second electrode.

69. The patch of claim 67, wherein said power supply comprises at least one thin and flexible electrochemical cell.

70. The patch of claim 67, wherein said patch is thin and flexible.

71. The patch of claim 67, wherein said patch is a printed patch.

72. The patch of claim 67, wherein said conductive layer is a hydrogel.

73. The patch of claim 67, wherein said first active electrode is in the shape of said body decoration and said conductive layer is in the shape of said body decoration.

74. The patch of claim 67, wherein said conductive layer comprises said color formulation.

75. The patch of claim 67, further comprising a decorative template that covers one or more regions of said body area and that is on said conductive formulation on said first electrode.

76. The patch of claim 75, wherein said decorative template is made of an insulated material.

77. The patch of claim 75, wherein said decorative template is made from polyester.

78. The patch of claim 75, wherein said decorative template comprises a cutout in the shape of said body decoration.

79. The patch of claim 75, wherein said decorative template limits application of said color formulation to those regions of said body area not covered by said template, and limits the electric current flowing from said first electrode to said body area to those regions of said body area not covered by said template.

80. The patch of claim 67, wherein said body decoration is a tattoo.

81. The patch of claim 67, wherein said body decoration is semi-permanent make-up.

82. An iontophoretic patch for applying a body decoration to a subject comprising:
(a) a first electrode, wherein said first electrode is an active electrode;
(b) a second electrode;
(c) a power supply attached to said electrodes for supplying current to said electrodes; and
(d) a color formulation on said first electrode;
wherein said patch promotes penetration of said color formulation into a body area of said subject to form said body decoration.

83. The iontophoretic patch of claim 82, further comprising a substrate base layer.

84. The iontophoretic patch of claim 82 further comprising a conductive layer disposed on said first electrode for providing a conductive interface between said first electrode and said body area.

85. The iontophoretic patch of claim 84, further comprising an additional conductive layer disposed on said second electrode for providing a conductive interface between said second electrode and said body area.

86. An iontophoretic patch for applying a body decoration to a body area of a subject and for treating said body area, comprising:
(a) a substrate base layer;
(b) a first electrode attached to said substrate base layer, wherein said first electrode is an active electrode;
(c) a second electrode attached to said substrate base layer;
(d) a power supply attached to said electrodes for supplying current to said electrodes;
(e) a conductive layer disposed on said first electrode for providing a conductive interface between said first electrode and said body area;
(f) a color formulation on said first electrode; and
(g) an active medicinal composition for treating said body area;
wherein said patch promotes penetration of said color formulation into said body area and promotes penetration of said active medicinal composition into said body area.

87. The patch of claim 86, further comprising an additional conductive layer disposed on said second electrode for providing a conductive interface between said second electrode and said body area.

88. The patch of claim 86, wherein said treating is selected from the group consisting of treatment of acne, wrinkles, skin discoloration, excessive skin coloration, skin puffiness, scarring, dry skin, oily skin, imbalance of skin pH, and a combination thereof.

89. A patch for removing a body decoration from a body area of a subject comprising:
(a) a substrate base layer;
(b) a first electrode attached to said substrate base layer, wherein said first electrode is an active electrode;
(c) a second electrode attached to said substrate base layer;
(d) a power supply for supplying current to said electrodes;
(e) a conductive layer disposed on said first electrode for providing a conductive interface between said first electrode and said body area; and
(f) a color formulation collecting chamber;
wherein said patch removes a color formulation from a body decoration and delivers said color formulation to said collecting chamber.

90. An iontophoretic patch for removing a body decoration from a body area of a subject comprising:
(a) a substrate base layer;
(b) a first electrode attached to said substrate base layer, wherein said first electrode is an active electrode;
(c) a second electrode attached to said substrate base layer;
(d) a power supply for supplying current to said electrodes;
(e) a conductive layer disposed on said first electrode for providing a conductive interface between said first electrode and said body area; and
(f) a removal formulation;
wherein said iontophoresis patch delivers said removal formulation to said body decoration to remove said body decoration.

91. A kit for applying a body decoration to a body area of a subject, comprising:
a color formulation; and
an electrically powered patch that promotes penetration of said color formulation into said body area.

92. The kit of claim 91, wherein said color formulation is applied to said body area by free-hand drawing.

93. A method of applying a body decoration to a subject, comprising the steps of:
contacting a body area of said subject with a means for applying;
applying a color formulation to said body area with said means for applying; and
promoting penetration of said color formulation into said body area with an electrically powered patch in contact with said formulation.

94. The method of claim 93, wherein said step of contacting a body area comprises adhering a stencil to said body area.

95. The method of claim 94, wherein said step of applying comprises applying said color formulation through said stencil to said body area.

96. The method of claim 93, wherein said step of contacting a body area comprises adhering a transferable sheet having said color formulation thereon to said body area.

97. The method of claim 96, wherein said step of applying comprises transferring said color formulation from said transferable sheet to said body area.

98. The method of claim 93, wherein said electrically powered patch is an iontophoretic patch.

99. The method of claim 96, wherein said transferable sheet comprises conductive elements configured to the shape of said body decoration.

100. The method of claim 99, wherein the resistance of said conductive elements is adjustable to control depth of penetration.

101. The method of claim 93, wherein said step of contacting a body area comprises contacting said body area with an active electrode in the shape of said body decoration, and wherein said electrode further comprises a conductive composition and a colored formulation in the shape of said body decoration.

102. The method of claim 93, wherein said step of promoting penetration comprises the use of an iontophoretic patch that provides an electric current to said body area after said step of applying said color formulation.

103. The method of claim 102, wherein said electric current is between from about 0.02 mAmp/cm² to about 0.5 mAmp/cm².

104. The method of claim 93, further comprising the step of: applying a pre-treatment to said body area prior to said step of contacting a body area.

105. The method of claim 104, wherein said pre-treatment is a peeling composition.

106. The method of claim 104, wherein said pre-treatment is a cleanser.

107. The method of claim 93, further comprising the step of: applying a sealant to said body area after said step of promoting penetration.

108. The method of claim 93, wherein said color formulation comprises an ink, the ink comprising colorant, a chemical base, and additives.

109. The method of claim 108, wherein said colorant comprises a dye, a pigment, or a combination thereof.

110. The method of claim 108, wherein said additives comprise at least one of a buffering agent, a resin, an adhesive, and a humectant.

111. The method of claim 93, wherein said body area comprises at least one of skin, nails, teeth, hair, and lips.

112. The method of claim 93, wherein said step of promoting penetration comprises contacting said body area with said patch for a time period up to about 10 hours.

113. The method of claim 93, wherein said step of promoting penetration comprises contacting said body area with said patch for a time period up to about 2 hours.

114. The method of claim 93, further comprising the step of selecting a type of color formulation, the colors of the color formulation and the design to be applied.

115. The method of claim 93, wherein said body decoration is a tattoo.

116. The method of claim 93, wherein said body decoration is semi-permanent make-up.

117. A method for applying a body decoration to a subject comprising the steps of:
(a) providing an iontophoretic patch for body decoration comprising:
(i) a first electrode, wherein said first electrode is an active electrode;
(ii) a second electrode;
(iii) a power supply for supplying current to said electrodes;
(iv) a conductive layer disposed on said first electrode for providing a conductive interface between said first electrode and a body area of said subject; and
(v) a color formulation on said first electrode;
wherein said patch promotes penetration of said color formulation into said body area to form said body decoration;
(b) contacting said body area with said iontophoretic patch for a time period wherein said patch promotes penetration of said color formulation into said body area to form said body decoration; and
(c) removing said iontophoretic patch from said body area.
